SEQUENCE LISTING (1 sequence)

SEQ ID NO: 1 - DNA, 1787 bp, unidentified
  source 1..1787: mol_type = genomic DNA
ctggttgattctgccagtagtcatacgcttgtctcaaagattaagccatgcatgtctcagtataaacaattatacagtgaaactgcgaacggctcattatatcagttatagtttctttgatagtgtttttctacatggatacttgtggcaaatctagaaacaatacatgcatcaaggcccgactttacggaagggccgcatttatttgacttaaaccaataccctttgggttgttttggtgattcagaataactaagcgaatcgcagtgccttcgggcggcgatgaatcattcaagtttctgccccatcagttgtcgatggtagggtattggcctaccatgactgttacgggtgacggagaattagggttcgattccggagagggagccttagagacggctaccacatccaaggaaggcagcaggcgcgtaaattactcaatgttaactcaacgaagtagtgacgagaaataacaatgccgagccctcagggtttggcaattggaatgagagcaatgtaaaaacctcatcgaggatcaattggagggcaagtctggtgccagcagccgcggtaattccagctccaatagcgtatattaaagttgttgcagttaaaaagctcgtagttgaacttctggtaggagtgacccggcctttgggcgaatgccctctgtttgctgtgtgtcatctctggccatcctcgctgtcttttaggcagcgtctttcactgtaaaaaaattagggtgtttcaagcaagattatttctggaatatattagtatgggatgataagataggctctcggtgctattttgttggtttgcacatcaagagaatgattaacagggacagttgggggtattcgtatttacatgtcagaggtgaaattcttggattttggaaagacgaactactgcgaaagcatttaccaaagatgttttcattaatcaagaacgaaagttaggggatcgaagatgattagataccatcgtagtcttaaccgtaaactatgccgacttgcgattgtccaatggttcttttagccgtgggcagcagcacatgagaaatcaaagtctttgggttccggggggagtatggtcgcaaggctgaaacttaaaggaattgacggaagggcaccaccaggagtggagcctgcggcttaatttgactcaacacgggaaaacttaccaggtccagacataggaaggattgacagattgagagctctttcttgattctatgggtggtggtgcatggccgttcttagttggtggagcgatttgtctggttaattccgttaacgaacgagaccacagcctactaaatagtatgtgctatggtaacatagtataagacttcttagagggacatttcgggtttaccggaaggaagtttgtggcaataacaggtctgtgatgcccttagatgttctgggccgcacgcgcgctacactgatgaattcaacgagttttgctctttgagcatttccttggccggaaggtctgggtaatcttttaaacgttcatcgtgatggggctagatttttgcaattattaatctccaacgaggaattcctagtaaacgcaagtcatcaacttgcattgattacgtccctgccctttgtacacaccgcccgtcgcacctaccgattgaacgttccggtgagaccttcggagcctgtgttttttcatttatttgagagttcacgggtaaagttgagcaaaccttaacgtttagaggaaggtgaagtcgtaacaaggtttccgtaggtgaacctgcagaaggatca

(19) United States
(12) Patent Application Publication
(10) Pub. No.: US 2026/0201427 A1
(43) Pub. Date: Jul. 16, 2026

(54) Novel Microalgae and Microalgal Compositions

(71) Applicant: MARA RENEWABLES CORPORATION, Dartmouth (CA)

(72) Inventors: David Woodhall, Dartmouth (CA); Kaitlyn Tanner, Dartmouth (CA); Jeremy Benjamin, Dartmouth (CA); Michael Milway, Dartmouth (CA); Michael Cheng, Dartmouth (CA); Denise Muise, Dartmouth (CA); Holly Rasmussen, Dartmouth (CA)

(73) Assignee: MARA RENEWABLES CORPORATION, Dartmouth, NS (CA)

(21) Appl. No.: 19/445,387

(22) Filed: Jan. 9, 2026

Related U.S. Application Data
(60) Provisional application No. 63/743,824, filed on Jan. 10, 2025.

(51) Int. Cl. C12P 7/6432; A23L 29/00; C12N 1/12; C12P 5/02; C12P 7/6434

(52) U.S. Cl. CPC C12P 7/6432; A23L 29/04; C12N 1/12; C12P 5/026; C12P 7/6434

(57) ABSTRACT

Provided herein are cultures comprising eukaryotic microorganisms having a lipid profile comprising eicosapentaenoic acid. Also provided are compositions comprising the eukaryotic microorganisms as well as methods of using the eukaryotic microorganisms.

### CROSS REFERENCE TO RELATED APPLICATION

This application claims priority to U.S. Provisional Application No. 63/723,824, filed Jan. 10, 2025, which is incorporated by reference herein in its entirety.

### SEQUENCE LISTING

The instant application contains a Sequence Listing that has been filed electronically in .xml format and is hereby incorporated by reference in its entirety. Said .xml copy, created on Jan. 8, 2026, is named MAR-023WO1 095523-1541046 Sequence Listing.xml and is 4000 bytes in size.

### BACKGROUND

Market demands require that new, sustainable sources of omega-3 fatty acids be developed. Omega-3 fatty acids derived from microorganisms are desirable for several reasons: 1) the source is vegetarian; 2) their production and pricing can be controlled to match demand; and 3) their production in a bioprocess produces fewer CO₂, emissions, and has less impact on the environment. Thraustochytrid microorganisms can be used to manufacture omega-3 fatty acids but do not tend to be abundant producers of eicosapentaenoic acid (20:5n-3, EPA). Thus, there is a need for microorganisms that can produce omega-3 fatty acids such as eicosapentaenoic acid (20:5n-3, EPA), docosapentaenoic acid (22:5 n-3, DPAn3), and docosahexaenoic acid (C22:6(n-3), DHA).

### BRIEF SUMMARY

Provided herein are cultures comprising a heterotrophic medium and lipid-producing eukaryotic microorganisms with an 18S sequence, wherein the 18S sequence has at least 90% identity to the sequence set forth in SEQ ID NO:1. Also provided is a lipid composition produced by and harvested from the eukaryotic microorganisms, wherein the lipid composition has a lipid profile comprising eicosapentaenoic acid (EPA) and/or docosahexaenoic acid (DHA), and/or docosapentaenoic acid n-3 (DPAn3). Also provided are compositions comprising the eukaryotic microorganisms. Methods of making a lipid composition using the disclosed eukaryotic microorganisms and methods of using the lipid compositions by incorporating the lipid compositions into foodstuffs are also provided herein. Also provided are methods of making a biomass using the disclosed microorganisms and optionally incorporating the biomass into foodstuffs.

### DETAILED DESCRIPTION

Microalgae are the primary producers of aquatic ecosystems and represent the origin of essential nutrients (e.g., omega-3 fatty acids) that are metabolized and/or bio-accumulated in the aquatic food chain. As such, high omega-3 fatty acid products derived from microalgae have enormous potential to sustainably meet the dietary demands of both the human and animal nutrition markets. Among the large variety of microalgae, heterotrophic microalgae (e.g., thraustochytrid microorganisms) have the greatest potential to provide aquaculture feed inputs that are free from the supply and demand constraints of plant and animal products. Heterotrophic microalgae production requires significantly less land and water, has better process economics, and is independent of environmental conditions (i.e., climate independent). Thraustochytrid microorganisms are often used in a commercial bioprocess to manufacture the omega-3 fatty acid DHA but do not tend to be abundant producers of EPA (generally, <2% of total fatty acids) or DPAn3. The present disclosure, however, provides Thraustochytrids that produce DHA, and/or EPA and/or DPAn3.

### Eukaryotic Microorganisms

Disclosed are eukaryotic microorganisms that produce oils or lipids. Microorganisms, including Thraustochytrids, produce a variety of lipids including fatty acids in various forms and amounts. As used herein, the term lipid includes phospholipids, free fatty acids, esters of fatty acids, triacylglycerols, sterols and sterol esters, carotenoids, xanthophylls (e.g., oxycarotenoids), hydrocarbons, and other lipids known to one of ordinary skill in the art. Fatty acids are hydrocarbon chains that terminate in a carboxyl group, being termed unsaturated if they contain at least one carbon-carbon double bond and polyunsaturated when they contain multiple carbon-carbon double bonds. Microorganisms can produce one or more of (i) short-chain fatty acids (SCFA), which are fatty acids with aliphatic tails of fewer than six carbons (e.g., butyric acid); (ii) medium-chain fatty acids (MCFA), which are fatty acids with aliphatic tails of 6-12 carbons; (iii) long-chain fatty acids (LCFA), which are fatty acids with aliphatic tails 13 to 21 carbons; and very long chain fatty acids (VLCFA), which are fatty acids with aliphatic tails 22 carbons or longer. Various microorganisms produce varying types and amounts of these fatty acids. The specific types and amounts of fatty acids produced in or by a microorganism or harvested from a microorganism are collectively referred to herein as the microorganism's lipid profile. Thus, as used herein, the term lipid profile refers to the types of lipids and amounts of lipids produced in or by a microorganism or harvested from a microorganism. By way of example, the lipid profile of a microorganism or lipid composition harvested from a microorganism can be determined by an analytical chemistry method, e.g., a FAMEs (fatty acid methyl esters) analysis.

Optionally, the lipid-producing or oil-producing microorganisms provided herein are of the Eukarya domain, Staminopila kingdom, Bigyra phylum, Labyrinthulomycota class, Thraustochytrida order, Thraustochytriaceae family, or Thraustochytrium genera. Optionally, the lipid-producing or oil-producing eukaryotic microorganisms have an 18S nucleic acid sequence with at least 90%, 91%, 92%, 93%, 94%, 95%, 96%, 97%, 98%, 99% or 100% identity to the nucleic acid sequence set forth in SEQ ID NO: 1. Optionally, the eukaryotic microorganisms are represented by microorganisms having IDAC Accession No. 210624-01, which were deposited with the International Depositary Authority of Canada (IDAC), National Microbiology Laboratory, Public Health Agency of Canada, 1015 Arlington Street, Winnipeg, Manitoba Canada R3E 3R2, on Jun. 21, 2024, and assigned Accession No. 210624-01. Optionally, the eukaryotic microorganisms are represented by microorganisms having IDAC Accession No. 161225-01, which were deposited with the International Depositary Authority of Canada (IDAC), National Microbiology Laboratory, Public Health Agency of Canada, 1015 Arlington Street, Winnipeg, Manitoba Canada R3E 3R2, on Dec. 16, 2025, and assigned Accession No. 161225-01. These deposits will be maintained under the terms of the Budapest Treaty on the International Recognition of the Deposit of Microorganisms for the Purposes of Patent Procedure. These deposits are exemplary and were made merely as a convenience for those of skill in the art and are not an admission that a deposit is required for patentability (e.g., under 35 U.S.C. § 112). The terms RT16 or RT16 strain or strain RT16 are used herein interchangeably to refer to the eukaryotic microorganisms described herein all of which have the 18S nucleic acid sequence set forth in SEQ ID NO:1.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more (or all) selected codons are substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Optionally, identity exists over a region that is at least about 25 amino acids or nucleotides in length or over a region that is 50-100 amino acids or nucleotides in length.

For sequence comparison, typically one sequence acts as a reference sequence, to which test sequences are compared. When using a sequence comparison algorithm, test and reference sequences are entered into a computer; subsequence coordinates are designated, if necessary; and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from about 20 to about 600, about 50 to about 200, or about 100 to about 150, in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, WI); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al.). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

The eukaryotic microorganisms disclosed herein produce lipids and have a lipid profile comprising fatty acids. Optionally, the lipid profile of the microorganism provided herein comprises 0%, 0.1%, 1%, 2%, 3%, or 4% to 19% of eicosapentaenoic acid (20:5n-3, EPA) by weight of total fatty acids. Optionally, the lipid profile comprises 0%, 0.1%, 1%, 2%, 3%, 4% to 6%, 5% to 7%, 7% to 9%, 8% to 10%, 9% to 11%, 10% to 12%, 11% to 13%, 12% to 14%, 13% to 15%, 15% to 17%, or 17% to 19% EPA by weight of total fatty acids. Optionally, the lipid profile of the microorganism provided herein comprises 0%, 0.1% or 1.0% to 45% docosahexaenoic acid (C22:6(n-3), DHA) by weight of total fatty acids. Optionally, the lipid profile comprises 0%, 0.1% or 1% to 5%, 5% to 10%, 10% to 15%, 15% to 20%, 20% to 25%, 25% to 30%, 30% to 35%, 35% to 40%, or 40% to 45% DHA by weight of total fatty acids. Optionally, the lipid profile comprises a ratio of EPA to DHA between 1:1 to 4:1. Optionally, the lipid profile comprises a ratio of EPA to DHA of 1:1, 2:1, 3:1, or 4:1.

Optionally, the lipid profile of the microorganism comprises docosapentaenoic acid (DPA). Optionally, the lipid profile comprises 0%, 0.1% or 1% to 6% docosapentaenoic acid (22:5 n-3, DPAn3) by weight of total fatty acids. Optionally, the lipid profile comprises 0%, 0.1% or 1% to 2%, 2% to 3%, 3% to 4%, 4%, to 5% or 5% to 6% DPAn3 by weight of total fatty acids. Optionally, the lipid profile comprises 0%, 0.1% or 1%, 1.5%, 2%, 2.5%, 3%, 3.5%, 4%, 4.5%, 5%, 5.5%, or 6% DPAn3 by weight of total fatty acids.

Optionally, the lipid profile of the microorganism comprises EPA, DHA, and DPAn3. Optionally, the lipid profile comprises 4% to 19% EPA, 1% to 45% DHA and 1% to 6% DPAn3. Optionally, the lipid profile comprises 4% to 10% EPA, 5% to 10% DHA, and 1% to 6% DP An3 by weight of total fatty acids. Optionally, the lipid profile comprises 4% to 10% EPA, 10% to 15% DHA, and 1% to 6% DPAn3 by weight of total fatty acids. Optionally, the lipid profile comprises 4% to 10% EPA, 15% to 20% DHA, and 1% to 6% DPAn3 by weight of total fatty acids. Optionally, the lipid profile comprises 4% to 10% EPA, 20 to 25% DHA, and 1% to 6% DPAn3 by weight of total fatty acids. Optionally, the lipid profile comprises 4% to 10% EPA, 25% to 30% DHA, and 2% to 6% DPAn3 by weight of total fatty acids. Optionally, the lipid profile comprises 4% to 10% EPA, 35% to 40% DHA, and 1% to 6% DPAn3 by weight of total fatty acids. Optionally, the lipid profile comprises 4% to 10% EPA, 40% to 45% DHA, and 1% to 6% DPAn3 by weight of total fatty acids.

Optionally, the lipid profile comprises 10% to 19% EPA, 5% to 10% DHA, and 1% to 6% DPAn3 by weight of total fatty acids. Optionally, the lipid profile comprises 10% to 19% EPA, 10% to 15% DHA, and 1% to 6% DPAn3 by weight of total fatty acids. Optionally, the lipid profile comprises 10% to 19% EPA, 15% to 20% DHA, and 1% to 6% DPAn3 by weight of total fatty acids. Optionally, the lipid profile comprises 10% to 19% EPA, 20 to 25% DHA, and 1% to 6% DPAn3 by weight of total fatty acids. Optionally, the lipid profile comprises 10% to 19% EPA, 25% to 30% DHA, and 1% to 6% DPAn3 by weight of total fatty acids. Optionally, the lipid profile comprises 10% to 19% EPA, 35% to 40% DHA, and 1% to 6% DPAn3 by weight of total fatty acids. Optionally, the lipid profile comprises 10% to 19% EPA, 40% to 45% DHA, and 1% to 6% DPAn3 by weight of total fatty acids.

Optionally, the lipid profile comprises 4% to 8% EPA, 5% to 10% DHA, and 1% to 4% DPAn3 by weight of total fatty acids. Optionally, the lipid profile comprises 4% to 8% EPA, 10% to 15% DHA, and 1% to 4% DPAn3 by weight of total fatty acids. Optionally, the lipid profile comprises 4% to 8% EPA, 15% to 20% DHA, and 1% to 4% DPAn3 by weight of total fatty acids. Optionally, the lipid profile comprises 4% to 8% EPA, 20 to 25% DHA, and 1% to 4% DPAn3 by weight of total fatty acids. Optionally, the lipid profile comprises 4% to 8% EPA, 25% to 30% DHA, and 1% to 4% DPAn3 by weight of total fatty acids. Optionally, the lipid profile comprises 4% to 8% EPA, 35% to 40% DHA, and 1% to 4% DPAn3 by weight of total fatty acids. Optionally, the lipid profile comprises 4% to 8% EPA, 40% to 45% DHA, and 1% to 4% DPAn3 by weight of total fatty acids.

Optionally, the lipid profile comprises 4% to 8% EPA, 5% to 10% DHA, and 4% to 6% DPAn3 by weight of total fatty acids. Optionally, the lipid profile comprises 4% to 8% EPA, 10% to 15% DHA, and 4% to 6% DPAn3 by weight of total fatty acids. Optionally, the lipid profile comprises 4% to 8% EPA, 15% to 20% DHA, and 4% to 6% DPAn3 by weight of total fatty acids. Optionally, the lipid profile comprises 4% to 8% EPA, 20 to 25% DHA, and 4% to 6% DPAn3 by weight of total fatty acids. Optionally, the lipid profile comprises 4% to 8% EPA, 25% to 30% DHA, and 4% to 6% DPAn3 by weight of total fatty acids. Optionally, the lipid profile comprises 4% to 8% EPA, 35% to 40% DHA, and 4% to 6% DPAn3 by weight of total fatty acids. Optionally, the lipid profile comprises 4% to 8% EPA, 40% to 45% DHA, and 4% to 6% DPAn3 by weight of total fatty acids.

Optionally, the lipid profile comprises 8% to 12% EPA, 5% to 10% DHA, and 1% to 4% DPAn3 by weight of total fatty acids. Optionally, the lipid profile comprises 8% to 12% EPA, 10% to 15% DHA, and 1% to 4% DPAn3 by weight of total fatty acids. Optionally, the lipid profile comprises 8% to 12% EPA, 15% to 20% DHA, and 1% to 4% DPAn3 by weight of total fatty acids. Optionally, the lipid profile comprises 8% to 12% EPA, 20 to 25% DHA, and 1% to 4% DPAn3 by weight of total fatty acids. Optionally, the lipid profile comprises 8% to 12% EPA, 25% to 30% DHA, and 1% to 4% DPAn3 by weight of total fatty acids. Optionally, the lipid profile comprises 8% to 12% EPA, 35% to 40% DHA, and 1% to 4% DPAn3 by weight of total fatty acids. Optionally, the lipid profile comprises 8% to 12% EPA, 40% to 45% DHA, and 1% to 4% DPAn3 by weight of total fatty acids.

Optionally, the lipid profile comprises 8% to 12% EPA, 5% to 10% DHA, and 4% to 6% DPAn3 by weight of total fatty acids. Optionally, the lipid profile comprises 8% to 12% EPA, 10% to 15% DHA, and 4% to 6% DPAn3 by weight of total fatty acids. Optionally, the lipid profile comprises 8% to 12% EPA, 15% to 20% DHA, and 4% to 6% DPAn3 by weight of total fatty acids. Optionally, the lipid profile comprises 8% to 12% EPA, 20 to 25% DHA, and 4% to 6% DPAn3 by weight of total fatty acids. Optionally, the lipid profile comprises 8% to 12% EPA, 25% to 30% DHA, and 4% to 6% DPAn3 by weight of total fatty acids. Optionally, the lipid profile comprises 8% to 12% EPA, 35% to 40% DHA, and 4% to 6% DPAn3 by weight of total fatty acids. Optionally, the lipid profile comprises 8% to 12% EPA, 40% to 45% DHA, and 4% to 6% DPAn3 by weight of total fatty acids.

Optionally, the lipid profile comprises 12% to 19% EPA, 5% to 10% DHA, and 1% to 4% DPAn3 by weight of total fatty acids. Optionally, the lipid profile comprises 12% to 19% EPA, 10% to 15% DHA, and 1% to 4% DPAn3 by weight of total fatty acids. Optionally, the lipid profile comprises 12% to 19% EPA, 15% to 20% DHA, and 1% to 4% DPAn3 by weight of total fatty acids. Optionally, the lipid profile comprises 12% to 19% EPA, 20 to 25% DHA, and 1% to 4% DPAn3 by weight of total fatty acids. Optionally, the lipid profile comprises 12% to 19% EPA, 25% to 30% DHA, and 1% to 4% DPAn3 by weight of total fatty acids. Optionally, the lipid profile comprises 12% to 19% EPA, 35% to 40% DHA, and 1% to 4% DPAn3 by weight of total fatty acids. Optionally, the lipid profile comprises 12% to 19% EPA, 40% to 45% DHA, and 1% to 4% DPAn3 by weight of total fatty acids.

Optionally, the lipid profile comprises 12% to 19% EPA, 5% to 10% DHA, and 4% to 6% DPAn3 by weight of total fatty acids. Optionally, the lipid profile comprises 12% to 19% EPA, 10% to 15% DHA, and 4% to 6% DPAn3 by weight of total fatty acids. Optionally, the lipid profile comprises 12% to 19% EPA, 15% to 20% DHA, and 4% to 6% DPAn3 by weight of total fatty acids. Optionally, the lipid profile comprises 12% to 19% EPA, 20 to 25% DHA, and 4% to 6% DPAn3 by weight of total fatty acids. Optionally, the lipid profile comprises 12% to 19% EPA, 25% to 30% DHA, and 4% to 6% DPAn3 by weight of total fatty acids. Optionally, the lipid profile comprises 12% to 19% EPA, 35% to 40% DHA, and 4% to 6% DPAn3 by weight of total fatty acids. Optionally, the lipid profile comprises 12% to 19% EPA, 40% to 45% DHA, and 4% to 6% DPAn3 by weight of total fatty acids. Optionally, the lipid profile comprises 4% to 10% EPA, 3% to 4% DPAn3 and 4% to 7% DHA by weight of total fatty acids.

Optionally, the lipid profile comprises, by weight of total fatty acids, 0.5% to 7.0% C14:0 (myristic acid) (e.g., 0.5% to 1.0%, 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5% or 6.5% to 7.0% of C14:0 (myristic acid)); 0% to 14% C15:0 (pentadecanoic acid) (e.g., 0% to 2%, 2% to 4%, 4% to 6%, 6% to 8%, 8% to 10%, 10% to 12%, or 12% to 14% of C15:0 (pentadecanoic acid)); 10% to 22% C16:0 (palmitic acid) (e.g., 10% to 12%, 12% to 14%, 14% to 16%, 16% to 18%, 18% to 20%, or 20% to 22% C16:0 (palmitic acid)); 2% to 32% C16:1 (palmitoleic acid) (e.g., 2% to 4%, 4% to 6%, 6% to 8%, 8% to 10%, 10% to 12%, 12% to 14%, 14% to 16%, 16% to 18%, 18% to 20%, 20% to 22%, 22% to 24%, 24% to 26%, 26% to 28%, 28% to 30%, or 30% to 32% C16:1 (palmitoleic acid)); 0% to 5% C17:0 (heptadecanoic acid) (e.g., 0.5% to 1.0%, 1.0% to 1.5%, 1.5% to 2.0%, 2.0% to 2.5%, 2.5% to 3.0%, 3.0% to 3.5%, 3.5% to 4.0%, 4.0% to 4.5%, or 4.5% to 5.0% C17:0 (heptadecanoic acid)); 1.0% to 6.0% C18:0 (stearic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, or 5.5% to 6.0%, C18:0 (stearic acid)); 3% to 35% C18:1 cis-9 (oleic acid) (e.g., 3% to 5%, 5% to 7%, 7%, to 9%, 9% to 11%, 11% to 13%, 13% to 15%, 15% to 17%, 17% to 19%, 19% to 21%, 21% to 23%, 23% to 25%, 25% to 27%, 27% to 29%, 29% to 31%, 31% to 33%, or 33% to 35% C18:1 cis-9 (oleic acid)); 5.0% to 35.0% C18:1 cis-7 (vaccenic acid) (e.g., 5% to 7%, 7%, to 9%, 9% to 11%, 11% to 13%, 13% to 15%, 15% to 17%, 17% to 19%, 19% to 21%, 21% to 23%, 23% to 25%, 25% to 27%, 27% to 29%, 29% to 31%, 31% to 33%, or 33% to 35% C18:1 cis-7 (vaccenic acid)); 4.0% to 10% C18:2 (n-6) (linoleic acid) (e.g., 4% to 6%, 6% to 8%, or 8% to 10% C18:2 (n-6) (linoleic acid)); 1.0% to 7.0% C18:3n-6 (gamma-Linolenic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5% or 6.5% to 7.0% C18:3n-6 (gamma-Linolenic acid)); 0% to 1.0% C20:2n-6 (Eicosadienoic acid) (e.g., 0% to 0.1%, 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, or 0.9% to 1.0% C20:2n-6 (Eicosadienoic acid)); 0.1% to 2.0% C20:3n-6 (Dihomo-γ-linolenic Acid) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, or 1.9% to 2.0% C20:3n-6 (Dihomo-γ-linolenic Acid)); 1.0% to 8.0% C20:4(n-6) (arachidonic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5%, 6.5% to 7.0%, 7.0% to 7.5%, or 7.5% to 8.0% C20:4(n-6) (arachidonic acid)); 0.1% to 4.0% C22:4n-6 (Adrenic Acid) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, 1.9% to 2.0%, 2.1% to 2.2%, 2.2% to 2.3%, 2.3% to 2.4%, 2.4% to 2.5%, 2.5% to 2.6%, 2.6% to 2.7%, 2.7% to 2.8%, 2.8% to 2.9%, 2.9% to 3.0%, 3.0% to 3.1%, 3.1% to 3.2%, 3.2% to 3.3%, 3.3% to 3.4%, 3.4% to 3.5%, 3.5% to 3.6%, 3.6% to 3.7%, 3.7% to 3.8%, 3.8% to 3.9%, or 3.9% to 4.0% C22:4n-6 (Adrenic Acid)); 4% to 19% C20:5n-3 (eicosapentaenoic acid, EPA) (e.g., 4% to 6%, 5% to 7%, 7% to 9%, 8% to 10%, 9% to 11%, 10% to 12%, 11% to 13%, 12% to 14%, 13% to 15%, 15% to 17%, or 17% to 19% C20:5n-3 (eicosapentaenoic acid, EPA)); 0.1% to 2.0% C22:5(n-6) (docosapentaenoic acid, DPAn6) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, or 1.9% to 2.0% C22:5(n-6) (docosapentaenoic acid, DPAn6); 1.0% to 6.0% C22:5(n-3)) (docosapentaenoic acid, DPAn3) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, or 5.5% to 6.0% C22:5(n-3)) (docosapentaenoic acid, DPAn3)); 1.0% to 45% C22:6(n-3) (docosahexaenoic acid, DHA) (e.g., 1% to 5%, 5% to 10%, 10% to 15%, 15% to 20%, 20% to 25%, 25% to 30%, 30% to 35%, 35% to 40%, or 40% to 45% C22:6(n-3) (docosahexaenoic acid, DHA)); or any combination of these fatty acids.

The provided microorganism has distinguishing features over wild type microorganisms in their natural environment. Wild type microorganisms can be found in natural aquatic environments extending from oceanic environments to freshwater lakes and rivers, and also include brackish environments such as estuaries and river mouths. Such environments are not considered to be encompassed by the term heterotrophic medium. The provided microorganisms produce different amounts of one or more lipids and/or protein content as compared to microorganisms in their natural environment, unexposed to laboratory or commercial production conditions. For example, the heterotrophic culture conditions described herein do not induce the significant production of a secreted ectoplasmic net since the adhesive, penetrative, and adsorptive properties of an ectoplasmic net are not required for the RT16 microorganisms to thrive in the industrial lipid-producing processes described herein. Ectoplasmic nets are secreted by microorganisms in their natural environment and surround the colony of microorganisms in that environment. Furthermore, since the RT16 microorganisms are grown in an abundance of carbon, the cells do not experience metabolic stress and the zoospore-producing, wild-type lifecycle stages are suppressed. In contrast to the RT16 microorganisms described herein, in nature, the wild-type RT16 microorganisms are more typically associated with growth on solid, decaying vegetative matter in 30% to 35% salinity seawater, at temperatures as low as 0 to 2 degrees Celsius, which are very different conditions from the laboratory or commercial environment.

The herein provided microorganisms can produce other components, for example, squalene. Optionally, the microorganism produces 10 mg to 30 mg of squalene per gram of biomass of the microorganisms. As used herein, biomass refers to the quantity or weight of microorganisms produced in a given space (e.g., volume or area) over a given period of time. Thus, a microorganism has a certain biomass referring to, for example, the total weight of the microorganisms. Optionally, the microorganisms produce 10 mg to 15 mg, 15 mg to 20 mg, 20 mg to 25 mg, 25 mg to 30 mg, 10 mg to 20 mg, 10 mg to 25 mg, 15 mg to 30 mg, 15 mg to 25 mg, or 20 mg to 30 mg of squalene per gram of biomass. Optionally, the microorganisms produce 10, 12, 14, 16, 18, 20, 22, 24, 26, 28, or 30 mg of squalene per gram of biomass.

### Cultures

Provided herein are cultures comprising the microorganisms described above and throughout the specification and a heterotrophic medium. Optionally, the lipid-producing or oil-producing microorganisms provided herein are of the Eukarya domain, Staminopila kingdom, Bigyra phylum, Labyrinthulomycota class, Thraustochytrida order, Thraustochytriaceae family, or Thraustochytrium genera. Optionally, the microorganism is a lipid-producing eukaryotic microorganism with an 18S sequence, wherein the 18S sequence has at least 90% identity to the sequence set forth in SEQ ID NO:1, wherein the microorganism produces fatty acids. Optionally, the lipid-producing or oil-producing eukaryotic microorganisms have an 18S nucleic acid sequence with at least 90%, 91%, 92%, 93%, 94%, 95%, 96%, 97%, 98%, 99% or 100% identity to the nucleic acid sequence set forth in SEQ ID NO: 1. Optionally, the eukaryotic microorganisms are represented by microorganisms having IDAC Accession No. 210624-01, which were deposited with the International Depositary Authority of Canada (IDAC), National Microbiology Laboratory, Public Health Agency of Canada, 1015 Arlington Street, Winnipeg, Manitoba Canada R3E 3R2, on Jun. 21, 2024, and assigned Accession No. 210624-01. Optionally, the eukaryotic microorganisms are represented by microorganisms having IDAC Accession No. 161225-01, which were deposited with the International Depositary Authority of Canada (IDAC), National Microbiology Laboratory, Public Health Agency of Canada, 1015 Arlington Street, Winnipeg, Manitoba Canada R3E 3R2, on Dec. 16, 2025, and assigned Accession No. 161225-01.

The heterotrophic medium used in the cultures provided herein supplies various nutritional components, including a carbon source and a nitrogen source, for the lipid-producing eukaryotic microorganism. Heterotrophic medium for the culture provided herein can include any of a variety of carbon sources. Examples of carbon sources include fatty acids, lipids, glycerols, triglycerols, carbohydrates, polyols, amino sugars, and any kind of biomass or waste stream. Fatty acids include, for example, oleic acid. Carbohydrates include, but are not limited to, glucose, cellulose, hemicellulose, fructose, dextrose (e.g., dextrose monohydrate), xylose, lactulose, galactose, maltotriose, maltose, lactose, glycogen, gelatin, starch (corn or wheat), acetate, m-inositol (e.g., derived from corn steep liquor), galacturonic acid (e.g., derived from pectin), L-fucose (e.g., derived from galactose), gentiobiose, glucosamine, alpha-D-glucose-1-phosphate (e.g., derived from glucose), cellobiose, dextrin, alpha-cyclodextrin (e.g., derived from starch), and sucrose (e.g., from molasses). Polyols include, but are not limited to, maltitol, erythritol, and adonitol. Amino sugars include, but are not limited to, N-acetyl-D-galactosamine, N-acetyl-D-glucosamine, and N-acetyl-beta-D-mannosamine. Optionally the carbon source is dextrose monohydrate. Optionally, the carbon source is glucose. Optionally, the carbon source is glycerol.

Optionally, the carbon source is present in the heterotrophic medium at a concentration of less than 60 g/L, including, for example, about 1 to about 60 g/L, about 5 to about 60 g/L, about 20 to about 40 g/L. Optionally the carbon source is present in the heterotrophic medium at a concentration of about 50 or about 55 g/L.

Optionally, the microorganisms are cultured in medium having a chloride concentration from about 0.5 g/L to about 50.0 g/L, including, for example, about 0.5 g/L to about 35 g/L, about 18 g/L to about 35 g/L, about 2 g/L to about 35 g/L. Optionally, the microorganisms described herein can be grown in low chloride conditions. For example, the microorganisms can be cultured in a medium having a chloride concentration from about 0.5 g/L to about 20 g/L (e.g., from about 0.5 g/L to about 15 g/L). The culture medium optionally includes NaCl. The culture medium can include non-chloride-containing sodium salts as a source of sodium. Examples of non-chloride sodium salts suitable for use in accordance with the present methods include, but are not limited to, soda ash (a mixture of sodium carbonate and sodium oxide), sodium carbonate, sodium bicarbonate, sodium sulfate, and mixtures thereof. See, e.g., U.S. Pat. Nos. 5,340,742 and 6,607,900, the entire contents of each of which are incorporated by reference herein. Optionally, the medium comprises 9 g/L chloride when using 20 g/L of carbon, 20 g/L soy peptone, and 5 g/L yeast extract. Optionally, the medium comprises 35 g/L chloride when the medium contains 10 g/L carbon, 5 g/L soy peptone, 5 g/L yeast extract and 10 g/L agar. Optionally, the medium comprises 2 g/L chloride when the medium contains 20-40 g/L carbon, 1 g/L yeast extract, 1-20 g/L monosodium glutamate (MSG), 0.3-2.0 g/L phosphates, 4 g/L magnesium sulfate, 5-10 g/L ammonium sulfate, 1.5 mL/L trace elements solution, 1 mL/L of vitamin B solution, 0.1 g/L CaCl₂).

Medium for a Thraustochytrid culture can include any of a variety of nitrogen sources. Exemplary nitrogen sources include ammonium solutions (e.g., NH₄ in H₂O), ammonium or amine salts (e.g., (NH₄)₂SO₄, (NH₄)₃PO₄, NH₄NO₃, NH₄CH₃CO₂ (NH₄Ac (ammonium acetate) peptone, soy peptone, tryptone, yeast extract, malt extract, fish meal, monosodium glutamate (MSG), soy extract, casamino acids and distiller grains. Optionally, the nitrogen source in the heterotrophic medium comprises at least one of yeast extract, monosodium glutamate (MSG), (NH₄)₂SO₄, urea, and NaNO₃. Optionally, the nitrogen source comprises yeast extract and at least one of (NH₄)₂SO₄, urea, or NaNO₃. Optionally, the nitrogen source comprises yeast extract. Optionally, the heterotrophic medium contains about 1.0 to about 30.0 g/L of a nitrogen source, including, for example, about 2.0 to 30.0 g/L, 3.0 to 30.0 g/L, 4.0 to 30.0 g/L, 5.0 to 30.0 g/L, 6.0 to 30.0 g/L, 7.0 to 30.0 g/L, 8.0 to 30.0 g/L, 9.0 to 30.0 g/L, 10.0 to 30.0 g/L, 11.0 to 30.0 g/L, 12.0 to 30.0 g/L, 13.0 to 30.0 g/L, 14.0 to 30.0 g/L, 15.0 to 30.0 g/L, 16.0 to 30.0 g/L, 17.0 to 30.0 g/L, 18.0 to 30.0 g/L, 19.0 to 30.0 g/L, 20.0 to 30.0 g/L, 21.0 to 30.0 g/L, 22.0 to 30.0 g/L, 23.0 to 30.0 g/L, 24.0 to 30.0 g/L, 25.0 to 30.0 g/L, 26.0 to 30.0 g/L, 27.0 to 30.0 g/L, 28.0 to 30.0 g/L, 29.0 to 30.0 g/L of a nitrogen source. Optionally, the heterotrophic medium comprises 1.0 to 3.4 g/L of a nitrogen source. Optionally, the nitrogen source comprises yeast extract, (NH₄)₂SO₄, urea, and NaNO₃.

The medium optionally includes a phosphate, such as potassium phosphate or sodium-phosphate. Optionally, the culture or heterotrophic medium comprises potassium phosphate monobasic.

Inorganic salts and trace nutrients may be present in the medium and may include ammonium sulfate, sodium bicarbonate, sodium orthovanadate, potassium chromate, sodium molybdate, selenous acid, nickel sulfate, copper sulfate, zinc sulfate, cobalt chloride, iron chloride, manganese chloride calcium chloride, and EDTA. Optionally, the medium includes at least 1.5 ml/L of a trace element solution. Optionally, the trace element solution comprises 2 mg/mL copper (II) sulfate pentahydrate, 2 mg/mL zinc sulfate heptahydrate, 1 mg/mL cobalt (II) chloride hexahydrate, 1 mg/mL manganese (II) chloride tetrahydrate, 1 mg/mL sodium molybdate dihydrate, and 1 mg/mL nickel (II) sulfate.

Optionally, the medium includes magnesium sulfate. Optionally, the heterotrophic medium or culture comprises magnesium sulfate, trace element solution, and potassium phosphate monobasic.

Vitamins such as pyridoxine hydrochloride, thiamine hydrochloride, calcium pantothenate, p-aminobenzoic acid, riboflavin, nicotinic acid, biotin, folic acid, and vitamin B12 can be included in the medium. Optionally, the heterotrophic medium contains vitamin B12 or lacks vitamin B12. Optionally, the vitamin B12 content in the medium is varied to control the production of pentadecanoic acid (C15:0). Optionally, a medium comprising a low or absent vitamin B12 content yields increased production of pentadecanoic acid. Optionally, a medium comprising a high vitamin B12 content yields decreased production of pentadecanoic acid (C15:0). Thus, for example, the lipid profile of microorganisms provided herein in a culture lacking vitamin B12 in the heterotrophic medium has a higher pentadecanoic acid (C15:0) content by weight of total fatty acids compared to a lipid profile of microorganisms of a control culture containing vitamin B12 in the heterotrophic medium. Optionally, the lipid profile of the microorganisms provided herein in a culture lacking vitamin B12 in the heterotrophic medium comprises 3% to 14% pentadecanoic acid (C15:0) content by weight of total fatty acids compared to a lipid profile of microorganisms of a control culture containing vitamin B12 in the heterotrophic medium. Control cultures typically contain 0% to 0.1% pentadecanoic acid (C15:0).

The pH of the medium can be adjusted to between and including 3.0 and 10.0 using acid or base, where appropriate, and/or using the nitrogen source. Optionally, the medium can be sterilized.

Generally, a medium used for culture of a microorganism is a liquid medium. However, the medium used for culture of a microorganism can be a solid medium. In addition to carbon and nitrogen sources as discussed herein, a solid medium can contain one or more components (e.g., agar and/or agarose) that provide structural support and/or allow the medium to be in solid form.

### Methods

### Methods of Making a Lipid Composition

Provided herein are methods of making a lipid composition comprising culturing the lipid-producing eukaryotic microorganism described herein in a heterotrophic medium to produce a lipid composition and isolating the lipid composition. For example, a method of making a lipid composition can include culturing lipid-producing eukaryotic microorganisms with an 18S sequence having at least 100% identity to the sequence set forth in SEQ ID NO: 1 in a heterotrophic medium; and isolating the lipid composition. Optionally, the method produces a lipid composition comprising 4% to 19% of EPA by weight of total fatty acids of the lipid composition. The methods may comprise culturing the lipid-producing eukaryotic microorganisms described herein in a heterotrophic medium to produce 0%, 0.1%, 1%, 2%, 3%, or 4% to 19% EPA by weight of total fatty acids. Optionally, the method produces a lipid composition comprising 4% to to 6%, 5% to 7%, 7% to 9%, 8% to 10%, 9% to 11%, 10% to 12%, 11% to 13%, 12% to 14%, 13% to 15%, 15% to 17%, or 17% to 19% EPA by weight of total fatty acids. Optionally, the lipid-producing eukaryotic microorganism used in the methods of making a lipid composition described herein are represented by microorganisms having IDAC Accession No. 210624-01. Optionally, the lipid-producing eukaryotic microorganism has an 18S nucleic acid sequence with at least 90%, 91%, 92%, 93%, 94%, 95%, 96%, 97%, 98%, 99% or 100% identity to the nucleic acid sequence set forth in SEQ ID NO: 1.

Optionally, the lipid composition comprises 0%, 0.1%, 1%, 2%, 3%, or 4% to 19% of eicosapentaenoic acid (20:5n-3, EPA) by weight of total fatty acids. Optionally, the lipid composition comprises 0%, 0.1%, 1%, 2%, 3%, 4% to 6%, 5% to 7%, 7% to 9%, 8% to 10%, 9% to 11%, 10% to 12%, 11% to 13%, 12% to 14%, 13% to 15%, 15% to 17%, or 17% to 19% EPA by weight of total fatty acids. Optionally, the lipid composition comprises 0%, 0.1% or 1.0% to 45% C22:6(n-3) (docosahexaenoic acid, DHA) by weight of total fatty acids. Optionally, the lipid composition comprises 0%, 0.1% or 1% to 5%, 5% to 10%, 10% to 15%, 15% to 20%, 20% to 25%, 25% to 30%, 30% to 35%, 35% to 40%, or 40% to 45% DHA by weight of total fatty acids. Optionally, the lipid composition comprises a ratio of EPA to DHA between 1:1 to 4:1. Optionally, the lipid composition comprises a ratio of EPA to DHA of 1:1, 2:1, 3:1, or 4:1.

Optionally, the lipid composition comprises docosapentaenoic acid (DPA). Optionally, the lipid composition comprises 1% to 6% docosapentaenoic acid (22:5 n-3, DPAn3) by weight of total fatty acids. Optionally, the lipid composition comprises 0%, 0.1% 1% to 2%, 2% to 3%, 3% to 4%, 4%, to 5% or 5% to 6% DPAn3 by weight of total fatty acids. Optionally, the lipid composition comprises 0%, 0.1%, 1%, 1.5%, 2%, 2.5%, 3%, 3.5%, 4%, 4.5%, 5%, 5.5%, or 6% DPAn3 by weight of total fatty acids.

Optionally, the lipid composition comprises EPA, DHA, and DPAn3. Optionally, the lipid composition comprises 4% to 19% EPA, 1% to 45% DHA and 1% to 6% DPAn3. Optionally, the lipid composition comprises 4% to 10% EPA, 5% to 10% DHA, and 1% to 6% DPAn3 by weight of total fatty acids. Optionally, the lipid composition comprises 4% to 10% EPA, 10% to 15% DHA, and 1% to 6% DPAn3 by weight of total fatty acids. Optionally, the lipid composition comprises 4% to 10% EPA, 15% to 20% DHA, and 1% to 6% DPAn3 by weight of total fatty acids. Optionally, the lipid composition comprises 4% to 10% EPA, 20 to 25% DHA, and 1% to 6% DPAn3 by weight of total fatty acids. Optionally, the lipid composition comprises 4% to 10% EPA, 25% to 30% DHA, and 2% to 6% DPAn3 by weight of total fatty acids. Optionally, the lipid composition comprises 4% to 10% EPA, 35% to 40% DHA, and 1% to 6% DPAn3 by weight of total fatty acids. Optionally, the lipid composition comprises 4% to 10% EPA, 40% to 45% DHA, and 1% to 6% DPAn3 by weight of total fatty acids.

Optionally, the lipid composition comprises 10% to 19% EPA, 5% to 10% DHA, and 1% to 6% DPAn3 by weight of total fatty acids. Optionally, the lipid composition comprises 10% to 19% EPA, 10% to 15% DHA, and 1% to 6% DPAn3 by weight of total fatty acids. Optionally, the lipid composition comprises 10% to 19% EPA, 15% to 20% DHA, and 1% to 6% DPAn3 by weight of total fatty acids. Optionally, the lipid composition comprises 10% to 19% EPA, 20 to 25% DHA, and 1% to 6% DPAn3 by weight of total fatty acids. Optionally, the lipid composition comprises 10% to 19% EPA, 25% to 30% DHA, and 1% to 6% DPAn3 by weight of total fatty acids. Optionally, the lipid composition comprises 10% to 19% EPA, 35% to 40% DHA, and 1% to 6% DPAn3 by weight of total fatty acids. Optionally, the lipid composition comprises 10% to 19% EPA, 40% to 45% DHA, and 1% to 6% DPAn3 by weight of total fatty acids.

Optionally, the lipid composition comprises 4% to 8% EPA, 5% to 10% DHA, and 1% to 4% DPAn3 by weight of total fatty acids. Optionally, the lipid composition comprises 4% to 8% EPA, 10% to 15% DHA, and 1% to 4% DPAn3 by weight of total fatty acids. Optionally, the lipid composition comprises 4% to 8% EPA, 15% to 20% DHA, and 1% to 4% DPAn3 by weight of total fatty acids. Optionally, the lipid composition comprises 4% to 8% EPA, 20 to 25% DHA, and 1% to 4% DPAn3 by weight of total fatty acids. Optionally, the lipid composition comprises 4% to 8% EPA, 25% to 30% DHA, and 1% to 4% DPAn3 by weight of total fatty acids. Optionally, the lipid composition comprises 4% to 8% EPA, 35% to 40% DHA, and 1% to 4% DPAn3 by weight of total fatty acids. Optionally, the lipid composition comprises 4% to 8% EPA, 40% to 45% DHA, and 1% to 4% DPAn3 by weight of total fatty acids.

Optionally, the lipid composition comprises 4% to 8% EPA, 5% to 10% DHA, and 4% to 6% DPAn3 by weight of total fatty acids. Optionally, the lipid composition comprises 4% to 8% EPA, 10% to 15% DHA, and 4% to 6% DPAn3 by weight of total fatty acids. Optionally, the lipid composition comprises 4% to 8% EPA, 15% to 20% DHA, and 4% to 6% DPAn3 by weight of total fatty acids. Optionally, the lipid composition comprises 4% to 8% EPA, 20 to 25% DHA, and 4% to 6% DPAn3 by weight of total fatty acids. Optionally, the lipid composition comprises 4% to 8% EPA, 25% to 30% DHA, and 4% to 6% DPAn3 by weight of total fatty acids. Optionally, the lipid composition comprises 4% to 8% EPA, 35% to 40% DHA, and 4% to 6% DPAn3 by weight of total fatty acids. Optionally, the lipid composition comprises 4% to 8% EPA, 40% to 45% DHA, and 4% to 6% DPAn3 by weight of total fatty acids.

Optionally, the lipid composition comprises 8% to 12% EPA, 5% to 10% DHA, and 1% to 4% DPAn3 by weight of total fatty acids. Optionally, the lipid composition comprises 8% to 12% EPA, 10% to 15% DHA, and 1% to 4% DPAn3 by weight of total fatty acids. Optionally, the lipid composition comprises 8% to 12% EPA, 15% to 20% DHA, and 1% to 4% DPAn3 by weight of total fatty acids. Optionally, the lipid composition comprises 8% to 12% EPA, 20 to 25% DHA, and 1% to 4% DPAn3 by weight of total fatty acids. Optionally, the lipid composition comprises 8% to 12% EPA, 25% to 30% DHA, and 1% to 4% DPAn3 by weight of total fatty acids. Optionally, the lipid composition comprises 8% to 12% EPA, 35% to 40% DHA, and 1% to 4% DPAn3 by weight of total fatty acids. Optionally, the lipid composition comprises 8% to 12% EPA, 40% to 45% DHA, and 1% to 4% DPAn3 by weight of total fatty acids.

Optionally, the lipid composition comprises 8% to 12% EPA, 5% to 10% DHA, and 4% to 6% DPAn3 by weight of total fatty acids. Optionally, the lipid composition comprises 8% to 12% EPA, 10% to 15% DHA, and 4% to 6% DPAn3 by weight of total fatty acids. Optionally, the lipid composition comprises 8% to 12% EPA, 15% to 20% DHA, and 4% to 6% DPAn3 by weight of total fatty acids. Optionally, the lipid composition comprises 8% to 12% EPA, 20 to 25% DHA, and 4% to 6% DPAn3 by weight of total fatty acids. Optionally, the lipid composition comprises 8% to 12% EPA, 25% to 30% DHA, and 4% to 6% DPAn3 by weight of total fatty acids. Optionally, the lipid composition comprises 8% to 12% EPA, 35% to 40% DHA, and 4% to 6% DPAn3 by weight of total fatty acids. Optionally, the lipid composition comprises 8% to 12% EPA, 40% to 45% DHA, and 4% to 6% DPAn3 by weight of total fatty acids.

Optionally, the lipid composition comprises 12% to 19% EPA, 5% to 10% DHA, and 1% to 4% DPAn3 by weight of total fatty acids. Optionally, the lipid composition comprises 12% to 19% EPA, 10% to 15% DHA, and 1% to 4% DPAn3 by weight of total fatty acids. Optionally, the lipid composition comprises 12% to 19% EPA, 15% to 20% DHA, and 1% to 4% DPAn3 by weight of total fatty acids. Optionally, the lipid composition comprises 12% to 19% EPA, 20 to 25% DHA, and 1% to 4% DPAn3 by weight of total fatty acids. Optionally, the lipid composition comprises 12% to 19% EPA, 25% to 30% DHA, and 1% to 4% DPAn3 by weight of total fatty acids. Optionally, the lipid composition comprises 12% to 19% EPA, 35% to 40% DHA, and 1% to 4% DPAn3 by weight of total fatty acids. Optionally, the lipid composition comprises 12% to 19% EPA, 40% to 45% DHA, and 1% to 4% DPAn3 by weight of total fatty acids.

Optionally, the lipid composition comprises 12% to 19% EPA, 5% to 10% DHA, and 4% to 6% DPAn3 by weight of total fatty acids. Optionally, the lipid composition comprises 12% to 19% EPA, 10% to 15% DHA, and 4% to 6% DPAn3 by weight of total fatty acids. Optionally, the lipid composition comprises 12% to 19% EPA, 15% to 20% DHA, and 4% to 6% DPAn3 by weight of total fatty acids. Optionally, the lipid composition comprises 12% to 19% EPA, 20 to 25% DHA, and 4% to 6% DPAn3 by weight of total fatty acids. Optionally, the lipid composition comprises 12% to 19% EPA, 25% to 30% DHA, and 4% to 6% DPAn3 by weight of total fatty acids. Optionally, the lipid composition comprises 12% to 19% EPA, 35% to 40% DHA, and 4% to 6% DPAn3 by weight of total fatty acids. Optionally, the lipid composition comprises 12% to 19% EPA, 40% to 45% DHA, and 4% to 6% DPAn3 by weight of total fatty acids. Optionally, the lipid profile comprises 4% to 10% EPA, 3% to 4% DPAn3 and 4% to 7% DHA by weight of total fatty acids.

Optionally, the lipid composition comprises, by weight of total fatty acids, 0.5% to 7.0% C14:0 (myristic acid) (e.g., 0.5% to 1.0%, 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5% or 6.5% to 7.0% of C14:0 (myristic acid)); 0% to 14% C15:0 (pentadecanoic acid) (e.g., 0% to 2%, 2% to 4%, 4% to 6%, 6% to 8%, 8% to 10%, 10% to 12%, or 12% to 14% of C15:0 (pentadecanoic acid)); 10% to 22% C16:0 (palmitic acid) (e.g., 10% to 12%, 12% to 14%, 14% to 16%, 16% to 18%, 18% to 20%, or 20% to 22% C16:0 (palmitic acid)); 2% to 32% C16:1 (palmitoleic acid) (e.g., 2% to 4%, 4% to 6%, 6% to 8%, 8% to 10%, 10% to 12%, 12% to 14%, 14% to 16%, 16% to 18%, 18% to 20%, 20% to 22%, 22% to 24%, 24% to 26%, 26% to 28%, 28% to 30%, or 30% to 32% C16:1 (palmitoleic acid)); 0% to 5% C17:0 (heptadecanoic acid) (e.g., 0.5% to 1.0%, 1.0% to 1.5%, 1.5% to 2.0%, 2.0% to 2.5%, 2.5% to 3.0%, 3.0% to 3.5%, 3.5% to 4.0%, 4.0% to 4.5%, or 4.5% to 5.0% C17:0 (heptadecanoic acid)); 1.0% to 6.0% C18:0 (stearic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, or 5.5% to 6.0%, C18:0 (stearic acid)); 3% to 35% C18:1 cis-9 (oleic acid) (e.g., 3% to 5%, 5% to 7%, 7%, to 9%, 9% to 11%, 11% to 13%, 13% to 15%, 15% to 17%, 17% to 19%, 19% to 21%, 21% to 23%, 23% to 25%, 25% to 27%, 27% to 29%, 29% to 31%, 31% to 33%, or 33% to 35% C18:1 cis-9 (oleic acid)); 5.0% to 35.0% C18:1 cis-7 (vaccenic acid) (e.g., 5% to 7%, 7%, to 9%, 9% to 11%, 11% to 13%, 13% to 15%, 15% to 17%, 17% to 19%, 19% to 21%, 21% to 23%, 23% to 25%, 25% to 27%, 27% to 29%, 29% to 31%, 31% to 33%, or 33% to 35% C18:1 cis-7 (vaccenic acid)); 4.0% to 10% C18:2 (n-6) (linoleic acid) (e.g., 4% to 6%, 6% to 8%, or 8% to 10% C18:2 (n-6) (linoleic acid)); 1.0% to 7.0% C18:3n-6 (gamma-Linolenic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5% or 6.5% to 7.0% C18:3n-6 (gamma-Linolenic acid)); 0% to 1.0% C20:2n-6 (Eicosadienoic acid) (e.g., 0% to 0.1%, 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, or 0.9% to 1.0% C20:2n-6 (Eicosadienoic acid)); 0.1% to 2.0% C20:3n-6 (Dihomo-γ-linolenic Acid) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, or 1.9% to 2.0% C20:3n-6 (Dihomo-γ-linolenic Acid)); 1.0% to 8.0% C20:4(n-6) (arachidonic acid) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5%, 6.5% to 7.0%, 7.0% to 7.5%, or 7.5% to 8.0% C20:4(n-6) (arachidonic acid)); 0.1% to 4.0% C22:4n-6 (Adrenic Acid) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, 1.9% to 2.0%, 2.1% to 2.2%, 2.2% to 2.3%, 2.3% to 2.4%, 2.4% to 2.5%, 2.5% to 2.6%, 2.6% to 2.7%, 2.7% to 2.8%, 2.8% to 2.9%, 2.9% to 3.0%, 3.0% to 3.1%, 3.1% to 3.2%, 3.2% to 3.3%, 3.3% to 3.4%, 3.4% to 3.5%, 3.5% to 3.6%, 3.6% to 3.7%, 3.7% to 3.8%, 3.8% to 3.9%, or 3.9% to 4.0% C22:4n-6 (Adrenic Acid)); 4% to 19% C20:5n-3 (eicosapentaenoic acid, EPA) (e.g., 4% to 6%, 5% to 7%, 7% to 9%, 8% to 10%, 9% to 11%, 10% to 12%, 11% to 13%, 12% to 14%, 13% to 15%, 15% to 17%, or 17% to 19% C20:5n-3 (eicosapentaenoic acid, EPA)); 0.1% to 2.0% C22:5(n-6) (docosapentaenoic acid, DPAn6) (e.g., 0.1% to 0.2%, 0.2% to 0.3%, 0.3% to 0.4%, 0.4%, to 0.5%, 0.5% to 0.6%, 0.6% to 0.7%, 0.7% to 0.8%, 0.8% to 0.9%, 0.9% to 1.0%, 1.0% to 1.1%, 1.1% to 1.2%, 1.2% to 1.3%, 1.3% to 1.4%, 1.4% to 1.5%, 1.5% to 1.6%, 1.6% to 1.7%, 1.7% to 1.8%, 1.8% to 1.9%, or 1.9% to 2.0% C22:5(n-6) (docosapentaenoic acid, DPAn6); 1.0% to 6.0% C22:5(n-3)) (docosapentaenoic acid, DPAn3) (e.g., 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, or 5.5% to 6.0% C22:5(n-3)) (docosapentaenoic acid, DPAn3)); 1.0% to 45% C22:6(n-3) (docosahexaenoic acid, DHA) (e.g., 1% to 5%, 5% to 10%, 10% to 15%, 15% to 20%, 20% to 25%, 25% to 30%, 30% to 35%, 35% to 40%, or 40% to 45% C22:6(n-3) (docosahexaenoic acid, DHA)); or any combination of these fatty acids.

Optionally, the cultured eukaryotic microorganisms have a biomass accumulation between 2.0 g/L to 150.0 g/L. Optionally, the cultured eukaryotic microorganisms have a biomass accumulation of 2.0 g/L to 150.0 g/L, such as 10.0 to 150 g/L, 15.0 to 150 g/L, 20.0 to 150 g/L, 25.0 to 150 g/L, 30.0 to 150 g/L, 35.0 to 150 g/L, 40.0 to 150 g/L, 45.0 to 150 g/L, 50.0 to 150 g/L, 55.0 to 150 g/L, 60.0 to 150 g/L, 65.0 to 150 g/L, 70.0 to 150 g/L, 75.0 to 150 g/L, 80.0 to 150 g/L, 85.0 to 150 g/L, 90.0 to 150 g/L, 95.0 to 150 g/L, 100.0 to 150 g/L, 105.0 to 150 g/L, 110.0 to 150 g/L, 115.0 to 150 g/L, 120.0 to 150 g/L, 125.0 to 150 g/L, 130.0 to 150 g/L, 135.0 to 150 g/L, 140.0 to 150 g/L, or 145.0 to 150 g/L. Biomass accumulation levels can be determined, for example, by X (gram)/Culture Volume (liter) with the final unit being g/L.

The heterotrophic medium used in the culturing step of the methods of making a lipid composition described herein may be the heterotrophic medium described herein throughout the specification. Thus, as described above, the heterotrophic medium used in the culture methods provided herein supplies various nutritional components, including a carbon source and a nitrogen source, for the lipid-producing eukaryotic microorganism. Optionally, the carbon source is glucose. Optionally, the carbon source is glycerol.

Optionally, the heterotrophic medium contains about 1.0 to about 30.0 g/L of a nitrogen source, including, for example, about 2.0 to 30.0 g/L, 3.0 to 30.0 g/L, 4.0 to 30.0 g/L, 5.0 to 30.0 g/L, 6.0 to 30.0 g/L, 7.0 to 30.0 g/L, 8.0 to 30.0 g/L, 9.0 to 30.0 g/L, 10.0 to 30.0 g/L, 11.0 to 30.0 g/L, 12.0 to 30.0 g/L, 13.0 to 30.0 g/L, 14.0 to 30.0 g/L, 15.0 to 30.0 g/L, 16.0 to 30.0 g/L, 17.0 to 30.0 g/L, 18.0 to 30.0 g/L, 19.0 to 30.0 g/L, 20.0 to 30.0 g/L, 21.0 to 30.0 g/L, 22.0 to 30.0 g/L, 23.0 to 30.0 g/L, 24.0 to 30.0 g/L, 25.0 to 30.0 g/L, 26.0 to 30.0 g/L, 27.0 to 30.0 g/L, 28.0 to 30.0 g/L, 29.0 to 30.0 g/L of a nitrogen source. Optionally, the nitrogen source in the heterotrophic medium comprises at least one of yeast extract, monosodium glutamate (MSG), (NH₄)₂SO₄, urea, and NaNO₃. Optionally, the nitrogen source comprises yeast extract and at least one of (NH₄)₂SO₄, urea, or NaNO₃. Optionally, the nitrogen source comprises yeast extract. Optionally, the heterotrophic medium comprises 1.0 to 3.4 g/L of a nitrogen source. Optionally, the nitrogen source comprises yeast extract, (NH₄)₂SO₄, urea, and NaNO₃.

Optionally, the heterotrophic medium contains vitamin B12 or lacks vitamin B12. Optionally, the vitamin B12 content in the medium is varied to control the production of pentadecanoic acid (C15:0). Optionally, a medium comprising a low or absent vitamin B12 content yields increased production of pentadecanoic acid. Optionally, a medium comprising a high vitamin B12 content yields decreased production of pentadecanoic acid (C15:0). Thus, for example, the lipid profile of microorganisms provided herein in a culture lacking vitamin B12 in the heterotrophic medium has a higher pentadecanoic acid (C15:0) content by weight of total fatty acids compared to a lipid profile of microorganisms of a control culture containing vitamin B12 in the heterotrophic medium.

Cultivation of the microorganisms can be carried out using known conditions, for example, those described in U.S. Pat. Nos. 10,676,775; 11,345,943; 10,920,261; and 11,827,918; and International Publication Nos. WO 2007/069078 and WO 2008/129358. For example, culturing can be carried out for 1 to 30 days, 1 to 21 days, 1 to 15 days, 1 to 12 days, 1 to 9 days, or 3 to 5 days. Optionally, culturing is carried out at temperatures between 4 to 30° C. Optionally, the culturing is carried out at temperatures between 15 to 25° C. or 15 to 20° C. or 20 to 25° C. Optionally, culturing is carried out by aeration-shaking culture, shaking culture, stationary culture, batch culture, continuous culture, rolling batch culture, wave culture, or the like. Optionally, culturing is carried out with a dissolved oxygen content of the culture medium between 0 and 80%, between 5 and 80%, between 10 and 80%, between 20 and 80%, between 30 and 80%, between 40 and 80%, between 50 and 80%, between 60 and 80%, or between 70 and 80%. The step of isolating the lipid composition may be performed using known methods.

### Methods of Using Lipid Compositions for Foodstuff

Optionally, the biomass or lipid compositions produced according to the methods described herein can be incorporated into a final product (e.g., a food or feed supplement, an infant formula, a pharmaceutical, a fuel, and the like). Thus, provided is a method of using a biomass or the lipid composition made according to the methods described herein, wherein the method of use comprises incorporating the lipid composition into a foodstuff. Optionally, the foodstuff is a human food or supplement, a pet food or supplement, a livestock feed or supplement, or an aquaculture feed or supplement.

Suitable food or feed supplements into which the lipid compositions can be incorporated include beverages, such as milk, water, sports drinks, energy drinks, teas, and juices; confections, such as candies, jellies, and biscuits; fat-containing foods and beverages, such as dairy products; processed food products, such as soft rice (or porridge); infant formulae; breakfast cereals; or the like. Optionally, the produced lipid compositions can be incorporated into a dietary supplement, such as, for example, a vitamin or multivitamin. Optionally, a lipid produced according to the method described herein can be included in a dietary supplement or optionally can be directly incorporated into a component of food or feed (e.g., a food supplement).

Examples of foodstuffs into which lipid compositions produced by the methods described herein can be incorporated include pet foods, such as cat foods; dog foods; feeds for aquarium fish, cultured fish or crustaceans, etc.; and feed for farm-raised animals (including livestock and fish or crustaceans raised in aquaculture). Food or feed material into which the lipid compositions produced according to the methods described herein can be incorporated is palatable to the organism that is the intended recipient. This food or feed material can have any physical properties currently known for a food material (e.g., solid, liquid, soft).

Optionally, one or more of the produced compounds (e.g., PUFAs) can be incorporated into a nutraceutical or pharmaceutical product. Examples of such a nutraceuticals or pharmaceuticals include various types of tablets, capsules, drinkable agents, etc. Optionally, the nutraceutical or pharmaceutical is suitable for topical application. Dosage forms containing the lipid compositions include, for example, capsules, oils, granula, granula subtilae, pulveres, tabellae, pilulae, trochisci, or the like.

The lipid compositions produced according to the methods described herein can be incorporated into products in combination with any of a variety of other agents. For instance, such compounds can be combined with one or more binders or fillers, chelating agents, pigments, salts, surfactants, moisturizers, viscosity modifiers, thickeners, emollients, fragrances, preservatives, etc., or any combination thereof.

### Isolated Biomass and Method of Making a Biomass

Provided herein are also isolated biomass comprising lipid-producing eukaryotic microorganisms with an 18S sequence, wherein the 18S sequence has at least 90% identity to the sequence set forth in SEQ ID NO:1, wherein the microorganisms in the biomass comprise fatty acids. Optionally, the lipid-producing or oil-producing eukaryotic microorganisms have an 18S nucleic acid sequence with at least 90%, 91%, 92%, 93%, 94%, 95%, 96%, 97%, 98%, 99% or 100% identity to the nucleic acid sequence set forth in SEQ ID NO: 1. Optionally, the eukaryotic microorganisms are represented by microorganisms having IDAC Accession No. 210624-01 or 161225-01. Optionally, the isolated biomass comprises 5 to 70 mg of EPA per gram of biomass. Optionally, the isolated biomass comprises 5 to 70, 10 to 70, 15 to 70, 20 to 70, 25 to 70, 30 to 70, 35 to 70, 40 to 70, 45 to 70, 50 to 70, 55 to 70, 60 to 70, or 65 to 70 mg of EPA per gram of biomass. Optionally, the isolated biomass comprises 1 to 40 mg of DPAn3 per gram of biomass. Optionally, the isolated biomass comprises 1 to 40 mg, 5 to 40 mg, 10 to 40 mg, 15 to 40 mg, 20 to 40 mg, 25 to 40, 30 to 40, or 35 to 40 mg of DPAn3 per gram of biomass. Optionally, the isolated biomass comprises 10 to 45 mg of DHA per gram of biomass. Optionally, the isolated biomass comprises 10 to 45, 15 to 45, 20 to 45, 25 to 45, 30 to 45, 35 to 45 or 40 to 45 mg of DHA per gram of biomass. Optionally, the isolated biomass comprises 5 to 70 mg of EPA and/or 1 to 40 mg of DPAn3 and/or 10 to 45 mg of DHA per gram of biomass.

Also provided are methods of making a biomass comprising culturing the lipid-producing eukaryotic microorganism described herein in a heterotrophic medium and isolating the biomass. The step of isolating the biomass may be performed using methods known in the art. Optionally, the lipid-producing eukaryotic microorganism used in the methods of making a biomass are represented by microorganisms having IDAC Accession No. 210624-01 or 161225-01. Optionally, the lipid-producing eukaryotic microorganism has an 18S nucleic acid sequence with at least 90%, 91%, 92%, 93%, 94%, 95%, 96%, 97%, 98%, 99% or 100% identity to the nucleic acid sequence set forth in SEQ ID NO:1. Optionally, the cultured eukaryotic microorganisms have a biomass accumulation of 2.0 g/L to 150.0 g/L, such as 10.0 to 150 g/L, 15.0 to 150 g/L, 20.0 to 150 g/L, 25.0 to 150 g/L, 30.0 to 150 g/L, 35.0 to 150 g/L, 40.0 to 150 g/L, 45.0 to 150 g/L, 50.0 to 150 g/L, 55.0 to 150 g/L, 60.0 to 150 g/L, 65.0 to 150 g/L, 70.0 to 150 g/L, 75.0 to 150 g/L, 80.0 to 150 g/L, 85.0 to 150 g/L, 90.0 to 150 g/L, 95.0 to 150 g/L, 100.0 to 150 g/L, 105.0 to 150 g/L, 110.0 to 150 g/L, 115.0 to 150 g/L, 120.0 to 150 g/L, 125.0 to 150 g/L, 130.0 to 150 g/L, 135.0 to 150 g/L, 140.0 to 150 g/L, or 145.0 to 150 g/L. Optionally, the isolated biomass comprises 5 to 70 mg of EPA per gram of biomass. Optionally, the isolated biomass comprises 5 to 70, 10 to 70, 15 to 70, 20 to 70, 25 to 70, 30 to 70, 35 to 70, 40 to 70, 45 to 70, 50 to 70, 55 to 70, 60 to 70, or 65 to 70 mg of EPA per gram of biomass. Optionally, the isolated biomass comprises 1 to 40 mg of DPAn3 per gram of biomass. Optionally, the isolated biomass comprises 1 to 40 mg, 5 to 40 mg, 10 to 40 mg, 15 to 40 mg, 20 to 40 mg, 25 to 40, 30 to 40, or 35 to 40 mg of DPAn3 per gram of biomass. Optionally, the isolated biomass comprises 10 to 45 mg of DHA per gram of biomass. Optionally, the isolated biomass comprises 10 to 45, 15 to 45, 20 to 45, 25 to 45, 30 to 45, 35 to 45 or 40 to 45 mg of DHA per gram of biomass. Optionally, the isolated biomass comprises 5 to 70 mg of EPA and/or 1 to 40 mg of DPAn3 and/or 10 to 45 mg of DHA per gram of biomass.

The heterotrophic medium used in the culturing step of the methods of making a biomass described herein may be the heterotrophic medium described herein. Optionally, the heterotrophic medium contains about 1.0 to about 30.0 g/L of a nitrogen source, including, for example, about 2.0 to 30.0 g/L, 3.0 to 30.0 g/L, 4.0 to 30.0 g/L, 5.0 to 30.0 g/L, 6.0 to 30.0 g/L, 7.0 to 30.0 g/L, 8.0 to 30.0 g/L, 9.0 to 30.0 g/L, 10.0 to 30.0 g/L, 11.0 to 30.0 g/L, 12.0 to 30.0 g/L, 13.0 to 30.0 g/L, 14.0 to 30.0 g/L, 15.0 to 30.0 g/L, 16.0 to 30.0 g/L, 17.0 to 30.0 g/L, 18.0 to 30.0 g/L, 19.0 to 30.0 g/L, 20.0 to 30.0 g/L, 21.0 to 30.0 g/L, 22.0 to 30.0 g/L, 23.0 to 30.0 g/L, 24.0 to 30.0 g/L, 25.0 to 30.0 g/L, 26.0 to 30.0 g/L, 27.0 to 30.0 g/L, 28.0 to 30.0 g/L, 29.0 to 30.0 g/L of a nitrogen source. Optionally, the nitrogen source in the heterotrophic medium comprises at least two of yeast extract, monosodium glutamate (MSG), (NH₄)₂SO₄, urea, and NaNO₃. Optionally, the nitrogen source comprises yeast extract, (NH₄)₂SO₄, urea, and NaNO₃. Optionally, the nitrogen source is yeast extract. Optionally, the heterotrophic medium comprises 1.0 to 3.4 g/L of a nitrogen source. Optionally, the nitrogen source comprises yeast extract, (NH₄)₂SO₄, urea, and NaNO₃.

Optionally, the method further comprises incorporating the biomass into a foodstuff as described herein. Optionally, the foodstuff is a human food or supplement, a pet food or supplement, a livestock feed or supplement, or an aquaculture feed or supplement. Thus, also provided is a method of using the biomass comprising incorporating the biomass into a foodstuff (e.g., a human food, pet food, a livestock feed, or an aquaculture feed).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### Example 1. Identification and Preliminary Analysis of RT16

Numerous strains of microorganisms were collected and tested under laboratory conditions for their ability to produce fatty acids in addition to DHA. Preliminary analysis indicated a strain designated RT16 was adapted to and designed to be able to accumulate high concentrations of oil rich in various relevant nutritional lipids under laboratory conditions. Due to these properties, RT16, identified as a Thraustochytrium species, was selected for further studies.

### Example 2. Ascertaining Optimal Temperature for Growth of RT16

A minimum temperature of 20° C. (i.e., about room temperature) is preferred to produce oils economically at scale. Therefore, a temperature experiment was conducted to determine the ability of the RT16 strain to grow at 20° C. Microorganisms were grown in media containing 20 g/L dextrose monohydrate, 13.75 g/L NaCl, 3.39 g/L MgSO₄·7H₂O, 2.69 g/L MgCl₂·6H₂O, 0.8 g/L CaCl₂·2H₂O, 3.21 g/L yeast extract, 0.26 g/L urea, 0.73 g/L NaNO₃, 0.57 g/L (NH₄)₂SO₄, 120 mg/L KH₂PO₄, 144 μg/L thiamine, 72 μg/L Ca-pantothenate, 72 μg/L nicotinic acid, 29 μg/L pyridoxine, 187 μg/L orotic acid, 36 μg/L biotin, 3.6 μg/L cobalamin (B12), 360 μg/L riboflavin, 720 μg/L pyridoxamine, 72 μg/L 4-aminobenzoic acid, 180 μg/L folic acid, 0.73 mg/L Na₂MoO₄·2H₂O, 2.3 mg/L NiSO₄·6H₂O, 24 mg/L FeCl₂·6H₂O, and 50 mM PIPES buffer (pH 6.5) at 15° C. and 20° C.

RT16 consumed 20 g/L of glucose in three days. Final pH was measured as 7.8, whereas initial pH, before sterilization, was 6.7. At 15° C., 6.23±0.39 g/L biomass was obtained, whereas, at 20° C., 5.57±0.7 g/L biomass was obtained. Total fatty acid produced was 460.37±6.2 mg/g and 460.86±8.16 mg/g, whereas the EPA percentage was 6.34 and 6.66% at 15° C. and 20° C., respectively. The ability of the microorganism to maintain its productivity and lipid composition under significantly different temperature conditions was unexpected, since regulation of lipid profile is a common means for microorganisms to maintain their membrane/metabolic fluidity (i.e., complex fatty acids are expected to be downregulated at higher temperatures). At 20° C., EPA productivity was found to be similar to productivity at 15° C., indicating that the energy requirements to chill the bioprocess were not significantly increased when growing the selected microorganisms at 20° C. Total fatty acid profile and EPA % can be seen in Table 1 below.

TABLE 1
Fatty acid methyl esters (FAMEs) profiles of RT16 grown at
15 and 20° C., expressed as percent of total fatty acids.

| FA (% of TFA) | 15° C. | 20° C. |
|---|---|---|
| C14:0 | 6.02 | 4.56 |
| C15:0 | 0.16 | 0.15 |
| C16:0 | 30.25 | 25.80 |
| C16:1 | 11.88 | 13.47 |
| C17:0 | 0.08 | 0.08 |
| C18:0 | 2.62 | 2.53 |
| C18:1n-9 | 7.75 | 9.83 |
| C18:1n-7 | 6.27 | 6.74 |
| C20:0 | 0.10 | 0.10 |
| C20:4n-6 | 2.54 | 2.96 |
| C20:5n-3 | 6.34 | 6.66 |
| C22:5n-6 | 0.31 | 0.27 |
| C22:5n-3 | 3.30 | 3.22 |
| C22:6n-3 | 7.51 | 7.11 |
| SFA (%) | 38.36 | 33.28 |
| MUFA (%) | 27.02 | 30.05 |
| PUFA (%) | 20.24 | 20.07 |
| Biomass (g/L) | 6.27 | 5.57 |
| TFA (mg/g) | 460.37 | 460.86 |

### Example 3. Optimizing Vitamin Components-Induction of Pentadecanoic Acid (C15:0) in the Lipid Profile

### Experimental Details

A full factorial, Placket-Burman design experiment was used to assess vitamin media ingredients essential for growth of the selected RT16 strain and the production of EPA and DHA. Minitab 18 software was used to design unique media formulations representing high- and low-levels for different media components. The low-level assigned as 0 g/L for any factor that was not known to be essential for growth and the high amount was set at the standard amount used in base media. The experiments were performed in 250 mL flasks with 60 mL of medium (50 g/L dextrose monohydrate, 1.9 g/L yeast extract, 3.39 MgSO₄·7H₂O, 13.75 g/L NaCl, 2.69 g/L MgCl₂·6H₂O, 1.3 g/L (NH₄)₂SO₄, 0.36 g/L KCl, 0.1 g/L NaHCO₃, 0.8 g/L CaCl₂): 2H₂O, 2.6 g/L MSG, 0.12 g/L KH₂PO₄, 4.8 mg/L NiSO₄·6H₂O, 24 mg/L FeSO₄·7H₂O, 7.2 mg/L MnCl₂·4H₂O, 7.2 mg/L ZnSO₄·7H₂O, 96 μug/L CoCl₂·6H₂O, 4.8 mg/L CuSO₄·5H₂O and 25 mM PIPES buffer (pH 6.5)). Flasks were inoculated with 5% of total volume of a 3-4 day old seed culture prior to reaching glucose depletion. Flasks were incubated at 20° C. with shaking at 200 rpm. Flasks were harvested when glucose dropped below 5 g/L after 4-14 days of incubation.

The specific vitamin components in the media formulations are described in Table 2 below. Following the incubation period, the biomass was collected, freeze-dried, and sent for FAMEs analysis.

### Results

FAMEs profiles for each of the conditions are shown in Table 3 below with the exception of 4, 8 and 18, which are duplicates of 10, 2 and 12, respectively. Of note, a deficiency in vitamin B12 induced the production of the odd-numbered carbon-chain fatty acid C15:0. Media compositions incorporating a deficiency in this vitamin could be used to control the production of the C15 fatty acid.

TABLE 2
Media formulations used in Example 3.

| | | Ca- | Nicotinic | | Orotic | | | | | | Folic |
|---|---|---|---|---|---|---|---|---|---|---|---|
| | Thiamine | Pantothenate | Acid | Pyridoxine | Acid | Biotin | B12 | Riboflavin | Pyridoxamine | P-Aminobenzoic | Acid |
| Flask | (g/L) | (μg/L) | (μg/L) | (μg/L) | (μg/L) | (μg/L) | (μg/L) | (μg/L) | (μg/L) | Acid (μg/L) | (μg/L) |
| 1 | 0 | 0 | 0 | 0 | 0 | 0 | 0 | 0 | 0 | 0 | 0 |
| 2 | 0 | 72 | 72 | 0 | 187.2 | 0 | 0 | 0 | 720 | 72 | 180 |
| 3 | 2 | 72 | 0 | 28.8 | 187.2 | 0 | 10000 | 0 | 0 | 0 | 180 |
| 4 | 0 | 0 | 0 | 28.8 | 187.2 | 10000 | 0 | 360 | 720 | 0 | 180 |
| 5 | 2 | 72 | 0 | 28.8 | 0 | 0 | 0 | 360 | 720 | 72 | 0 |
| 6 | 2 | 0 | 72 | 0 | 0 | 0 | 10000 | 360 | 720 | 0 | 180 |
| 7 | 2 | 0 | 72 | 28.8 | 0 | 10000 | 0 | 0 | 0 | 72 | 180 |
| 8 | 0 | 72 | 72 | 0 | 187.2 | 0 | 0 | 0 | 720 | 72 | 180 |
| 9 | 2 | 0 | 0 | 0 | 187.2 | 10000 | 10000 | 0 | 720 | 72 | 0 |
| 10 | 0 | 0 | 0 | 28.8 | 187.2 | 10000 | 0 | 360 | 720 | 0 | 180 |
| 11 | 2 | 72 | 0 | 28.8 | 0 | 0 | 0 | 360 | 720 | 72 | 0 |
| 12 | 0 | 72 | 0 | 0 | 0 | 10000 | 10000 | 360 | 0 | 72 | 180 |
| 13 | 0 | 72 | 72 | 28.8 | 0 | 10000 | 10000 | 0 | 720 | 0 | 0 |
| 14 | 2 | 0 | 0 | 0 | 187.2 | 10000 | 10000 | 0 | 720 | 72 | 0 |
| 15 | 2 | 72 | 0 | 28.8 | 187.2 | 0 | 10000 | 0 | 0 | 0 | 180 |
| 16 | 2 | 0 | 72 | 28.8 | 0 | 10000 | 0 | 0 | 0 | 72 | 180 |
| 17 | 0 | 0 | 72 | 28.8 | 187.2 | 0 | 10000 | 360 | 0 | 72 | 0 |
| 18 | 0 | 72 | 0 | 0 | 0 | 10000 | 10000 | 360 | 0 | 72 | 180 |
| 19 | 2 | 72 | 72 | 0 | 187.2 | 10000 | 0 | 360 | 0 | 0 | 0 |
| 20 | 0 | 0 | 0 | 0 | 0 | 0 | 0 | 0 | 0 | 0 | 0 |
| 21 | 0 | 0 | 72 | 28.8 | 187.2 | 0 | 10000 | 360 | 0 | 72 | 0 |
| 22 | 2 | 0 | 72 | 0 | 0 | 0 | 10000 | 360 | 720 | 0 | 180 |
| 23 | 0 | 72 | 72 | 28.8 | 0 | 10000 | 10000 | 0 | 720 | 0 | 0 |
| 24 | 2 | 72 | 72 | 0 | 187.2 | 10000 | 0 | 360 | 0 | 0 | 0 |

TABLE 3
FAMEs profiles of RT16 microorganisms grown in the media formulations
of Table 2, expressed as percent of total fatty acids.

| Flask | 1 | 20 | 2 | 3 | 15 | 5 | 11 | 6 | 22 | 7 | 16 |
|---|---|---|---|---|---|---|---|---|---|---|---|
| C14:0 | 1.3 | 1.3 | 1.3 | 4.5 | 4.5 | 0.8 | 0.7 | 4.7 | 4.5 | 6.1 | 6.7 |
| C15:0 | 13.6 | 5.1 | 12.2 | 0.0 | 0.1 | 4.2 | 7.2 | 0.0 | 0.1 | 4.2 | 4.2 |
| C16:0 | 15.2 | 16.7 | 15.7 | 19.1 | 19.5 | 15.5 | 15.6 | 18.9 | 19.1 | 17.7 | 17.5 |
| C16:1 | 4.2 | 5.1 | 4.6 | 27.1 | 27.8 | 2.5 | 2.7 | 29.3 | 27.4 | 23.2 | 23.8 |
| C17:0 | 4.6 | 2.2 | 4.0 | 0.0 | 0.0 | 2.7 | 4.5 | 0.0 | 0.0 | 1.5 | 1.4 |
| C18:0 | 1.3 | 1.8 | 1.4 | 2.0 | 1.9 | 1.4 | 1.4 | 2.1 | 1.8 | 1.1 | 1.0 |
| C18:1 Ole | 5.5 | 5.9 | 5.8 | 10.9 | 12.0 | 5.5 | 5.3 | 11.4 | 12.8 | 3.9 | 3.8 |
| C18:1 Vac | 6.8 | 7.2 | 7.3 | 6.2 | 5.9 | 5.2 | 6.1 | 6.2 | 6.1 | 6.4 | 6.4 |
| C18:2(n-6) | 5.5 | 6.7 | 5.7 | 9.9 | 8.6 | 4.2 | 5.0 | 9.1 | 8.5 | 8.9 | 8.8 |
| C18:3n-6 | 1.9 | 2.6 | 2.0 | 3.9 | 3.7 | 2.0 | 1.6 | 3.4 | 3.7 | 6.0 | 6.3 |
| C20:2n-6 | 0.5 | 0.3 | 0.5 | 0.1 | 0.1 | 0.5 | 0.6 | 0.1 | 0.1 | 0.1 | 0.1 |
| C20:3n-6 | 0.8 | 1.1 | 0.8 | 0.7 | 0.5 | 1.0 | 0.9 | 0.6 | 0.6 | 0.8 | 0.8 |
| C20:4(n-6) | 2.9 | 6.6 | 2.9 | 2.2 | 1.9 | 3.9 | 3.1 | 1.9 | 1.9 | 2.8 | 2.6 |
| C20:5(n-3) | 7.8 | 10.7 | 7.9 | 3.0 | 2.8 | 13.4 | 10.5 | 2.7 | 2.8 | 4.0 | 3.9 |
| EPA | | | | | | | | | | | |
| C22:4n-6 | 3.2 | 2.4 | 3.1 | 2.2 | 2.1 | 3.4 | 3.3 | 1.8 | 2.1 | 3.1 | 2.9 |
| C22:5(n-6) | 0.4 | 1.5 | 0.4 | 0.4 | 0.4 | 1.1 | 0.5 | 0.4 | 0.4 | 0.3 | 0.2 |
| DPA | | | | | | | | | | | |
| C22:5(n-3) | 5.2 | 2.7 | 4.7 | 2.2 | 2.1 | 4.2 | 4.5 | 2.0 | 2.0 | 3.6 | 3.5 |
| DPA | | | | | | | | | | | |
| C22:6(n-3) | 17.3 | 18.4 | 17.7 | 4.7 | 5.0 | 26.5 | 24.3 | 4.4 | 5.0 | 4.9 | 4.6 |
| DHA | | | | | | | | | | | |
| Other | 2.0 | 1.8 | 1.9 | 1.0 | 1.1 | 2.0 | 2.2 | 1.0 | 1.1 | 1.4 | 1.4 |
| % oil | 20.3 | 22.3 | 20.1 | 66.1 | 59.4 | 13.3 | 14.8 | 66.6 | 61.3 | 64.0 | 66.5 |
| biomass g/L | 3.2 | 0.5 | 3.1 | 14.1 | 10.0 | 3.4 | 3.4 | 16.1 | 10.8 | 17.2 | 16.8 |

| Flask | 9 | 14 | 10 | 12 | 13 | 23 | 17 | 21 | 19 | 24 |
|---|---|---|---|---|---|---|---|---|---|---|
| C14:0 | 4.5 | 4.7 | 7.0 | 4.4 | 4.6 | 4.6 | 4.5 | 4.4 | 6.5 | 6.5 |
| C15:0 | 0.0 | 0.1 | 3.4 | 0.0 | 0.1 | 0.0 | 0.0 | 0.1 | 4.5 | 3.7 |
| C16:0 | 19.7 | 19.4 | 17.1 | 19.1 | 20.8 | 19.1 | 20.2 | 21.2 | 17.6 | 17.0 |
| C16:1 | 26.8 | 28.8 | 27.5 | 30.2 | 30.6 | 30.4 | 29.0 | 29.2 | 23.7 | 24.9 |
| C17:0 | 0.0 | 0.0 | 1.1 | 0.0 | 0.0 | 0.0 | 0.0 | 0.1 | 1.5 | 1.2 |
| C18:0 | 2.1 | 2.1 | 1.0 | 1.5 | 1.9 | 1.6 | 1.6 | 2.1 | 1.0 | 1.0 |
| C18:1 Ole | 10.9 | 11.1 | 4.2 | 12.0 | 10.7 | 12.5 | 10.2 | 11.2 | 3.5 | 4.0 |
| C18:1 Vac | 5.9 | 5.9 | 6.1 | 6.7 | 5.8 | 6.3 | 6.9 | 5.8 | 6.7 | 6.3 |
| C18:2(n-6) | 9.8 | 9.5 | 7.6 | 8.1 | 7.5 | 8.0 | 7.9 | 7.1 | 8.6 | 9.0 |
| C18:3n-6 | 4.1 | 3.7 | 6.0 | 3.5 | 3.8 | 3.6 | 4.2 | 3.7 | 6.1 | 6.1 |
| C20:2n-6 | 0.1 | 0.1 | 0.1 | 0.1 | 0.1 | 0.1 | 0.1 | 0.1 | 0.1 | 0.1 |
| C20:3n-6 | 0.6 | 0.6 | 0.7 | 0.6 | 0.6 | 0.6 | 0.6 | 0.5 | 0.8 | 0.8 |
| C20:4(n-6) | 2.3 | 1.9 | 2.4 | 1.8 | 1.7 | 1.8 | 2.1 | 1.7 | 2.7 | 2.7 |
| C20:5(n-3) | 3.0 | 2.6 | 3.5 | 2.5 | 2.2 | 2.3 | 2.7 | 2.4 | 3.9 | 3.9 |
| EPA | | | | | | | | | | |
| C22:4n-6 | 2.1 | 2.1 | 2.7 | 2.0 | 2.3 | 2.0 | 2.2 | 2.3 | 3.0 | 3.0 |
| C22:5(n-6) | 0.3 | 0.3 | 0.2 | 0.4 | 0.3 | 0.3 | 0.3 | 0.3 | 0.3 | 0.2 |
| DPA | | | | | | | | | | |
| C22:5(n-3) | 2.2 | 2.2 | 3.4 | 2.0 | 2.3 | 2.0 | 2.3 | 2.3 | 3.5 | 3.5 |
| DPA | | | | | | | | | | |
| C22:6(n-3) | 4.5 | 4.1 | 4.4 | 4.1 | 3.9 | 3.8 | 4.3 | 4.4 | 4.6 | 4.5 |
| DHA | | | | | | | | | | |
| Other | 1.0 | 1.0 | 1.4 | 1.0 | 0.9 | 1.0 | 1.0 | 0.9 | 1.5 | 1.4 |
| % oil | 66.3 | 68.9 | 65.6 | 70.3 | 71.3 | 73.6 | 69.8 | 64.7 | 65.8 | 69.2 |
| biomass g/L | 14.0 | 13.4 | 16.5 | 15.7 | 14.6 | 15.4 | 14.0 | 11.2 | 16.8 | 16.4 |

### Example 4. Bioreactor Process Producing EPA from RT16 Microorganisms

### Experimental Details

Microorganisms of the strain designated RT16 were pre-cultured in baffled, 2 L Erlenmeyer flasks containing 500 mL of liquid media (55 g/L dextrose monohydrate, 2 g/L yeast extract, 3.4 MgSO₄·7H₂O, 2 g/L NaCl, 2.7 g/L MgCl₂·6H₂O, 2.65 g/L monosodium glutamate, 1.33 g/L (NH₄)₂SO₄, 0.8 g/L CaCl₂): 2H₂O, 144 μg/L thiamine, 72 μg/L Ca-pantothenate, 72 μg/L nicotinic acid, 29 μg/L pyridoxine, 187 μg/L orotic acid, 36 μg/L biotin, 3.6 μg/L cobalamin (B12), 360 μg/L riboflavin, 720 μg/L pyridoxamine, 72 μg/L 4-aminobenzoic acid, 180 μg/L folic acid, 0.73 mg/L Na₂MoO₄·2H₂O, 2.3 mg/L NiSO₄·6H₂O, 24 mg/L FeCl₃·6H₂O, and 50 mM PIPES buffer (pH 6.5)). Flasks were incubated under agitation at 20° C. and 200 rpm for 4 days. After the incubation period, 350 mL of the pre-culture were transferred into 3.15 L (10% v/v) of a media containing 55 g/L dextrose monohydrate, 9.7 g/L yeast extract, 3.4 g/L MgSO₄·7H₂O, 2 g/L NaCl, 2.7 g/L MgCl₂·6H₂O, 12.9 g/L monosodium glutamate, 6.44 g/L (NH₄)₂SO₄, 0.8 g/L CaCl₂): 2H₂O, 144 μg/L thiamine, 72 μg/L Ca-pantothenate, 72 μg/L nicotinic acid, 29 μg/L pyridoxine, 187 μg/L orotic acid, 36 μg/L biotin, 3.6 μg/L cobalamin (B12), 360 μg/L riboflavin, 720 μg/L pyridoxamine, 72 μg/L 4-aminobenzoic acid, 180 μg/L folic acid, 7.2 mg/L MnCl₂·4H₂O, 7.2 mg/L ZnSO₄·7H₂O, 0.096 mg/L CoCl₂·6H₂O, 4.8 mg/L CuSO₄·5H₂O, 4.8 mg/L NiSO₄·6H₂O, 24 mg/L FeSO₄·7H₂O in a 5-L fermentation vessel.

Batch culture conditions were applied as follows: 20° C., agitation starting at 200 rpm and reaching 540 rpm, aeration at 0.5 VVM with atmospheric air (fixed and based on the starting volume), and pH 6.4-6.6. Fed-batch cultures were carried out for a total of 261 hours. Cultures were fed with a 75% dextrose solution using a slow continuous pump rate between 0.06 mL/min and 2 mL/min to maintain a ≤20 g/L background dextrose concentration. At the third day, cultures were sampled twice daily to monitor glucose concentration, biomass accumulation, total fatty acids (TFA), and lipid profile of RT16.

### Results

RT16 microorganisms produced 73.8 g/L biomass and 76.9% TFA. EPA (5.7%), DHA (3.9%), and DPA (n-3) (3.4%) were the main PUFAs produced (Table 4). Thus, for this fermentation, RT16 microorganisms produced 43.8 mg of EPA per gram of biomass. These data showed RT16 microorganisms are capable of commercial production of EPA.

TABLE 4
Fatty acid profile of RT16 cultured in a bioreactor.

| FA | Value % |
|---|---|
| 14:0 | 5.1 |
| 15:0 | 4.9 |
| 16:0 | 14.1 |
| 16:1 | 20.3 |
| 17:0 | 1.7 |
| 17:1 | 2.5 |
| 18:0 | 0.9 |
| 18:1 oleic acid | 5.9 |
| 18:1 vaccenic acid | 8.6 |
| 18:2 (n-6) | 6.7 |
| 18:3 (n-6) | 3.9 |
| 20:3 (n-6) | 0.9 |
| 20:4 (n-6) | 4.3 |
| 20:5 (n-3) EPA | 5.7 |
| 22:4 (n-6) | 2.7 |
| 22:5 (n-6) DPA | 0.2 |
| 22:5 (n-3) DPA | 3.4 |
| 22:6 (n-3) DHA | 3.9 |

### Example 5. Growth in Glycerol Enhances EPA Productivity by RT16 Microorganisms

### Experimental Details

Microorganisms of strain RT16 were pre-cultured in liquid media (50 g/L dextrose monohydrate, 3.2 g/L yeast extract, 3.4 MgSO₄·7H₂O, 2 g/L NaCl, 2.7 g/L MgCl₂·6H₂O, 0.27 g/L urea, 0.6 g/L (NH₄)₂SO₄, 0.8 g/L CaCl₂): 2H₂O, 0.73 g/L NaNO₃, 0.12 g/L KH₂PO₄, 144 μg/L thiamine, 72 μg/L Ca-pantothenate, 72 μg/L nicotinic acid, 29 μg/L pyridoxine, 187 μg/L orotic acid, 36 μg/L biotin, 3.6 μg/L cobalamin (B12), 360 μg/L riboflavin, 720 μg/L pyridoxamine, 72 μg/L 4-aminobenzoic acid, 180 μg/L folic acid, 4.8 mg/L NiSO₄·6H₂O, 24 mg/L FeCl₃·6H₂O, 7.2 mg/L MnCl₂·4H₂O, 7.2 mg/L ZnSO₄·7H₂O, 96 μg/L CoCl₂·6H₂O, 4.8 mg/L CuSO₄·5H₂O and 50 mM MOPS buffer (pH 7.0)) until the concentration of dextrose monohydrate remaining was 1-20 g/L (i.e., not starving). 3 mL of this culture were used to inoculate 75 mL of the above medium (EXCEPT dextrose monohydrate), but containing one of the following conditions: (1) no carbon source; (2)₂₀ g/L dextrose monohydrate; (3)₂₀ g/L glycerol; (4)₂₀ g/L D-fructose; (5)₂₀ g/L D-galactose; (6)₂₀ g/L D-xylose; (7)₂₀ g/L D-mannitol; (8)₂₀ g/L D-sorbitol; (9) 20 g/L sucrose; (10) 33 g/L glucose and 17 g/L glycerol. These conditions 1-10 are listed in the first column of Table 5 below. The RT16 strain did not accumulate further biomass from the inoculate provided to the culture under conditions (1) no carbon source, (6)₂₀ g/L D-xylose, (7) 20 g/L D-mannitol, (8)₂₀ g/L D-sorbitol, and (9)₂₀ g/L sucrose. Cultures were incubated for 7 days at 20° C., 200 rpm.

### Results

FAMEs profiles, as a percent of Total Fatty Acids (TFA), for each of the conditions are shown in Table 5 below. The percentage of EPA in the lipid profile of the RT16 microorganisms increased from 8.72 when grown in dextrose to 13.98 when grown in glycerol, whilst maintaining a similar biomass (8.6 g/L and 8.9 g/L, respectively) and lipid content (35.54% and 32.61%, respectively). The increased EPA productivity displaced MUFA productivity (33.36% and 27.21%, respectively). These data showed that glycerol can be used in a fermentation process to specifically induce the production that favors EPA over other lipid species.

TABLE 5
FAMEs profiles of RT16 grown in glycerol as a percent of TFA.

| | | | | | | | | | | C20:5 | C22:5 | C22:5 | C22:6 | | bio- |
|---|---|---|---|---|---|---|---|---|---|---|---|---|---|---|---|
| Condi- | | | | | C18:1 | C18:1 | C18:2 | C18:3 | C20:4 | n-3 | n-6 | n-3 | n-3 | % | mass |
| tion | C14:0 | C16:0 | C16:1 | C18:0 | Oleic | Vaccenic | n-6 | n-6 | n-6 | EPA | DPA | DPA | DHA | oil | g/L |
| 1 | 1.49 | 14.83 | 8.93 | 1.06 | 5.25 | 8.68 | 2.69 | 1.31 | 2.95 | 15.70 | 1.41 | 0.78 | 31.31 | 10.78 | 1.04 |
| 2 | 2.23 | 16.50 | 12.94 | 2.02 | 12.19 | 8.23 | 8.15 | 4.13 | 4.74 | 8.72 | 0.85 | 2.77 | 10.66 | 35.54 | 8.60 |
| 3 | 1.67 | 17.14 | 8.47 | 1.88 | 8.67 | 10.07 | 7.19 | 4.15 | 7.19 | 13.98 | 0.53 | 2.90 | 10.31 | 32.61 | 8.91 |
| 4 | 2.16 | 20.78 | 6.28 | 1.02 | 12.93 | 7.23 | 5.41 | 2.61 | 3.42 | 10.51 | 0.84 | 2.93 | 18.42 | 19.68 | 5.93 |
| 5 | 1.32 | 20.89 | 3.37 | 0.96 | 8.34 | 7.55 | 4.46 | 0.86 | 3.03 | 15.01 | 0.80 | 1.09 | 28.69 | 13.39 | 3.33 |
| 6 | 1.12 | 13.87 | 4.79 | 1.03 | 3.03 | 4.78 | 2.50 | 0.77 | 2.77 | 18.51 | 1.94 | 1.09 | 40.59 | 8.92 | 0.83 |
| 7 | 1.33 | 14.88 | 5.70 | 0.97 | 3.99 | 5.99 | 2.67 | 0.99 | 2.82 | 17.57 | 1.70 | 0.93 | 37.26 | 9.10 | 0.95 |
| 8 | 1.49 | 14.81 | 8.22 | 1.05 | 4.62 | 7.50 | 2.60 | 1.07 | 2.91 | 16.04 | 1.75 | 0.84 | 33.50 | 9.81 | 1.15 |
| 9 | 1.51 | 15.19 | 9.09 | 1.07 | 5.07 | 8.30 | 3.05 | 1.22 | 2.98 | 15.09 | 1.48 | 0.82 | 31.77 | 10.15 | 1.20 |
| 10 | 2.45 | 17.40 | 12.62 | 0.95 | 4.26 | 8.26 | 5.97 | 2.29 | 3.99 | 6.29 | 0.34 | 3.31 | 6.76 | 51.73 | 10.71 |

### Example 6. Optimizing Nitrogen Source for RT16

### Experimental Details

Combinations of five alternative nitrogen sources, shown in Table 6 below, were tested to find the optimal combination to support growth of RT16 microorganisms. Nitrogen sources (g/L) were adjusted to reflect the nitrogen content of each compound and to maintain an equal total amount of nitrogen across conditions. The culture media used in the experiments included in different combinations, yeast extract, monosodium glutamate (MSG), (NH₄)₂SO₄, urea, and NaNO₃, in a base medium consisting of 50 g/L glucose, 13.75 g/l NaCl, 3.39 g/L MgSO₄, 2.69 g/L MgCl₂, 0.36 g/L KCl, 0.1 g/L NaHCO₃, and 0.8 g/L CaCl₂).

TABLE 6
Nitrogen source combinations.

| Flask | Yeast Extract | MSG | (NH₄)₂SO₄ | Urea | NaNO₃ |
|---|---|---|---|---|---|
| 1 | 0 | 0 | 0 | 3.4 | 0 |
| 2 | 0 | 3.4 | 0 | 0 | 0 |
| 3 | 0 | 0 | 0 | 0 | 3.4 |
| 4 | 0.34 | 0.34 | 0.34 | 2.04 | 0.34 |
| 5 | 3.4 | 0 | 0 | 0 | 0 |
| 6 | 0 | 0 | 0 | 0 | 3.4 |
| 7 | 3.4 | 0 | 0 | 0 | 0 |
| 8 | 0.68 | 0.68 | 0.68 | 0.68 | 0.68 |
| 9 | 0 | 0 | 3.4 | 0 | 0 |
| 10 | 2.04 | 0.34 | 0.34 | 0.34 | 0.34 |
| 11 | 0.68 | 0.68 | 0.68 | 0.68 | 0.68 |
| 12 | 0.34 | 0.34 | 0.34 | 0.34 | 2.04 |
| 13 | 0.34 | 2.04 | 0.34 | 0.34 | 0.34 |
| 14 | 0 | 0 | 3.4 | 0 | 0 |
| 15 | 0.34 | 2.04 | 0.34 | 0.34 | 0.34 |
| 16 | 0.34 | 0.34 | 2.04 | 0.34 | 0.34 |
| 17 | 0.34 | 0.34 | 0.34 | 2.04 | 0.34 |
| 18 | 0 | 0 | 0 | 3.4 | 0 |
| 19 | 0.34 | 0.34 | 2.04 | 0.34 | 0.34 |
| 20 | 2.04 | 0.34 | 0.34 | 0.34 | 0.34 |
| 21 | 0 | 3.4 | 0 | 0 | 0 |
| 22 | 0.34 | 0.34 | 0.34 | 0.34 | 2.04 |

### Results

Fatty acid profiles for each nitrogen source combination are shown in Table 7 below. Flasks containing MSG as the only carbon source or as the highest source of nitrogen neither supported growth nor any significant consumption of glucose. The pH of these flasks was also very basic. Yeast extract was the only nitrogen ingredient that was necessary for growth, as the flasks containing (NH₄)₂SO₄, urea, or NaNO₃ did not grow. However, if (NH₄)₂SO₄, urea, or NaNO₃ were present as the highest source of nitrogen and the media contained yeast extract, there was comparable growth when all five ingredients were present at equal levels. When yeast extract was the major nitrogen source, the flask accumulated the highest amount of biomass. Therefore, a useful nitrogen composition consisted of yeast extract and a minor combination of (NH₄)₂SO₄, urea, and NaNO₃.

TABLE 7
Fatty acid profiles in varying nitrogen source combinations.

| | | Biomass | TFA | EPA | EPA |
|---|---|---|---|---|---|
| Condition | pH | (g/L) | (%) | (%) | (mg/g) |
| Equal All | 6.46 ± 0.03 | 23.70 ± 0.16 | 23.76 ± 0.42 | 8.52 ± 0.54 | 20.22 ± 0.91 |
| High YE | 6.75* | 27.47 ± 2.01 | 17.69 ± 3.33 | 13.58 ± 0.86 | 23.86 ± 2.99 |
| Only YE | 6.52 ± 0.15 | 24.74 ± 0.39 | 29.45 ± 0.27 | 5.54 ± 0.20 | 16.31 ± 0.44 |
| High MSG | 9.08 ± 0.07 | 3.60 ± 0.35 | 4.06 ± 0.49 | 12.58 ± 1.33 | 5.14 ± 1.15 |
| Only MSG | 9.12 ± 0.014 | 3.98 ± 0.11 | 15.07 ± 1.51 | 8.26 ± 0.53 | 12.42 ± 0.45 |
| High | 4.96 ± 0.42 | 18.49 ± 0.33 | 45.08 ± 0.49 | 4.22 ± 0.13 | 19.04 ± 0.38 |
| (NH₄)₂SO₄ | | | | | |
| Only | 4.68* | 3.11 ± 1.28 | 19.05 ± 1.46 | 8.47 ± 3.03 | 16.37 ± 7.00 |
| (NH₄)₂SO₄ | | | | | |
| High Urea | 6.54 ± 0.02 | 21.72 ± 1.44 | 29.41 ± 1.40 | 5.60 ± 0.02 | 15.45 ± 0.73 |
| Only Urea | 8.265 ± 0.02 | 3.14 ± 0.13 | 22.60 ± 3.20 | 9.30 ± 0.51 | 20.90 ± 1.82 |
| High | 6.63 ± 0.03 | 21 ± 1.76 | 41.10 ± 0.54 | 4.23 ± 0.14 | 17.38 ± 0.33 |
| NaNO₃ | | | | | |
| Only | 6.73* | 2.05 ± 1.08 | 24.56 ± 0.07 | 8.74 ± 0.69 | 21.46 ± 1.76 |
| NaNO₃ | | | | | |

### Example 7. Growth of RT16 Microorganisms in Vessels

### Experimental Details

Cultures of microorganisms of strain RT16 were grown in two 7 L bioreactors, A and B. Media for bioreactor A contained 50 g/L glucose, 9.66 g/L yeast extract, 13.75 g/L NaCl, 2.69 g/L MgCl₂, 3.39 g/L MgSO₄, 0.36 g/L KCl, 0.1 g/L NaHCO₃, 0.8 g/L CaCl₂), 12.87 g/L monosodium glutamate, 6.44 g/L (NH₄)₂SO₄ and 2.4 g/L KH₂PO₄ with trace vitamins and minerals. The media for bioreactor B contained 50 g/L glucose, 2 g/L yeast extract, 1.65 g/L NaCl, 3.08 g/L MgSO₄, 0.39 g/L FeCl₃, 15.42 g/L (NH₄)₂SO₄, 1.67 g/L KH₂PO₄, 1.82 g/L K₂HPO₄, 0.5 g/L CaCl₂) and trace vitamins and minerals. The bioreactor conditions were maintained at 20° C., pH 6.5, 0.5 volume of air per unit volume of growth medium per minute oxygen (vvm) and agitated at 400 rpm.

### Results

Analysis of the resulting biomass is shown in Table 8 below.

TABLE 8
Biomass profiles of RT16 microorganisms grown in vessels.

| Vessel | A | B |
|---|---|---|
| Time (h) | 261 | 261 |
| Biomass (g/L) | 83 | 50 |
| TFA (mg/g) | 648 | 417 |
| SFA (%) | 29 | 21 |
| MUFA (%) | 35 | 44 |
| DHA (%) | 5.2 | 6.7 |
| EPA (%) | 4.6 | 6.3 |

### Example 8. Quantification of Squalene Content in Lipid Compositions Produced by RT16 Microorganisms

### Experimental Details

Biomass samples of thraustochytrid strains T18 (ATCC Accession No. PTA-6245), G3 (IDAC Accession No. 220716-01), and RT16 microorganisms were analysed for squalene content according to the method of Budge and Barry, MethodsX, 6:15-21 (2009).

### Results

Squalene content of the microorganisms is shown in Table 9 below. The squalene content of the RT16 strain is significantly higher than the amounts found in the two other thraustochytrid strains. These data indicate that the RT16 strain possesses the metabolic molecular machinery required for squalene production, when grown under conditions optimized for lipid productivity, and has the potential to be used as a platform biotechnology for the commercial production of squalene.

TABLE 9
Squalene content analysis.

| Strain | Squalene (mg/g biomass) |
|---|---|
| RT16 | 20.1 |
| T18 | 0.5 |
| G3 | 0.4 |

### Example 9. 100% Glycerol Validations

Cultures of microorganisms of strain RT16 were pre-cultured in baffled, 2 L Erlenmeyer flasks containing 500 mL of liquid media (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.36 g/L KCl, 0.1 g/L NaHCO₃, 0.8 g/L CaCl₂): 2H₂O, 0.12 g/L KH₂PO₄, 0.73 mg/L Na₂MoO₄·2H₂O, 2.3 mg/L NiSO₄·6H₂O, 24 mg/L FeCl₃·6H₂O, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 3.6 μg/L cobalamin (B12)). Flasks were incubated at 20° C. with 200 rpm of agitation for 4 days.

After the incubation period, 350 mL of each pre-culture was transferred into 3.15 L (10% v/v) of a media containing: 50 g/L glycerol, and 9.66 g/L yeast extract (Leiber E), 3.4 g/L MgSO₄·7H₂O, 4 g/L NaCl, 2.7 g/L MgCl₂·6H₂O, 6.44 g/L (NH₄)₂SO₄, 12.87 g/L monosodium glutamate, 0.8 g/L CaCl₂): 2H₂O, 0.12 g/L KH₂PO₄, 7.2 mg/L MnCl₂·4H₂O, 7.2 mg/L ZnSO₄·7H₂O, 96 μg/L CoCl₂·6H₂O, 4.8 mg/L CuSO₄·5H₂O, 4.8 mg/L NiSO₄·6H₂O, 24 mg/L FeSO₄·7H₂O, 2.4 mg/L Citric acid (anhydrous), 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, and 3.6 μg/L cobalamin (B12), in a 5 L fermentation vessel.

Batch culture conditions were applied as follows: 20° C., agitation between 200 and 800 rpm controlled by a 30% DO cascade, aeration at 0.5 VVM (fixed and based on the starting volume) with atmospheric air, and pH 6.4-6.6. Fed-batch cultures were carried out for a total of 238 hours. Cultures were fed with a 100% glycerol solution, using a slow continuous pump rate between 0.06 mL/min and 2 mL/min to maintain ≤20 g/L background glycerol concentration. Starting on the third day, samples were taken daily to monitor glycerol concentration, biomass accumulation, TFA, and oil profile.

After 238 hours, RT16 in vessel one consumed 264 g/L glycerol and produced 95.0 g/L biomass and 63.4% TFA. EPA (5.9%), DPAn-3 (3.8%), and DHA (4.3%) were the omega 3 PUFA produced (Table 10 below). RT16 in vessel two consumed 257 g/L glycerol and produced 90.9 g/L biomass and 62.9% TFA. EPA (6.1%), DPAn-3 (4.1%), and DHA (4.5%) were the omega 3 PUFA produced (Table 10 below). RT16 in vessel three consumed 273 g/L glycerol and produced 95.3 g/L biomass and 65.1% TFA. EPA (5.8%), DPAn-3 (3.9%), and DHA (4.2%) were the omega 3 PUFA produced (Table 10 below). RT16 in vessel four consumed 260 g/L glycerol and produced 93.2 g/L biomass and 62.9% TFA. EPA (5.8%), DPAn-3 (3.8%), and DHA (4.4%) were the omega 3 PUFA produced (Table 10 below).

These fermentations showed great reproducibility of the process and validated the ability to hit the goals of 55 g/L oil and 5.5% EPA. The average g/L oil±standard deviation was 59.5±2.1, and the average % EPA was 5.9±0.1 across the four fermentations.

TABLE 10
Fatty acid profiles expressed as percent of total fatty acids.

| Fatty | Vessel 1 | Vessel 2 | Vessel 3 | Vessel 4 |
|---|---|---|---|---|
| Acid | (Value %) | (Value %) | (Value %) | (Value %) |
| C14:0 | 4.7 | 4.5 | 4.8 | 4.7 |
| C15:0 | 1.5 | 2.2 | 1.2 | 1.5 |
| C16:0 | 18.5 | 19.9 | 18.7 | 18.6 |
| C16:1 | 16.9 | 15.8 | 17.6 | 16.6 |
| C17:0 | 0.5 | 0.7 | 0.4 | 0.5 |
| C17:1 | 0.8 | 1.0 | 0.6 | 0.8 |
| C17:2 | 0.1 | 0.3 | 0.1 | 0.2 |
| C18:0 | 1.1 | 1.1 | 1.1 | 1.1 |
| C18:1n-9 | 9.7 | 6.8 | 9.1 | 10.4 |
| C18:1n-7 | 11.0 | 11.0 | 11.3 | 11.1 |
| C18:2n-6 | 8.7 | 7.8 | 8.4 | 8.5 |
| C18:3n-6 | 3.6 | 4.1 | 3.8 | 3.4 |
| C20:3n-6 | 1.1 | 1.2 | 1.1 | 1.1 |
| C20:4n-6 | 3.2 | 3.8 | 3.2 | 3.0 |
| C20:5n-3 | 5.9 | 6.1 | 5.8 | 5.8 |
| C22:4n-6 | 2.0 | 2.5 | 2.1 | 2.0 |
| C22:5n-3 | 3.8 | 4.1 | 3.9 | 3.8 |
| C22:6n-3 | 4.3 | 4.5 | 4.2 | 4.4 |

### Example 10. Impact of the Two Yeast Extracts with Two Different Carbon Sources, Glucose and Glycerol, on RT16 Fermentation Performance for Production of EPA Containing Oil

Cultures of microorganisms of strain RT16 were pre-cultured in baffled, 2 L Erlenmeyer flasks containing 500 mL of liquid media (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.36 g/L KCl, 0.1 g/L NaHCO₃, 0.8 g/L CaCl₂): 2H₂O, 0.12 g/L KH₂PO₄, 0.73 mg/L Na₂MoO₄·2H₂O, 2.3 mg/L NiSO₄·6H₂O, 24 mg/L FeCl₃·6H₂O, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 3.6 μg/L cobalamin (B12)). Flasks were incubated at 20° C. with 200 rpm of agitation for 4 days.

After the incubation period, 350 mL of the pre-culture was transferred into 3.15 L (10% v/v) of a media containing: 3.4 g/L MgSO₄·7H₂O, 4 g/L NaCl, 2.7 g/L MgCl₂·6H₂O, 6.44 g/L (NH₄)₂SO₄, 12.87 g/L monosodium glutamate, 0.8 g/L CaCl₂): 2H₂O, 0.12 g/L KH₂PO₄, 7.2 mg/L MnCl₂·4H₂O, 7.2 mg/L ZnSO₄·7H₂O, 96 μg/L CoCl₂·6H₂O, 4.8 mg/L CuSO₄·5H₂O, 4.8 mg/L NiSO₄·6H₂O, 24 mg/L FeSO₄·7H₂O, 2.4 mg/L Citric acid (anhydrous), 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, and 3.6 μg/L cobalamin (B12), in a 5 L fermentation vessel.

In addition to the above-described media, vessel one contained: 50 g/L glucose, and 9.66 g/L Leiber E yeast extract; vessel two contained: 50 g/L glycerol, and 9.66 g/L Leiber E yeast extract; vessel three contained: 50 g/L glucose, and 9.66 g/L Leiber H yeast extract; and vessel four contained: 50 g/L glycerol, and 9.66 g/L Leiber H yeast extract.

Batch culture conditions were applied as follows: 20° C., agitation between 200 and 800 rpm controlled by a 30% DO cascade, aeration at 0.5 VVM (fixed and based on the starting volume) with atmospheric air, and pH 6.4-6.6. Fed-batch cultures were carried out for a total of 191-238 hours. Cultures were fed with either a 75% glucose or a 100% glycerol solution, using a slow continuous pump rate between 0.06 mL/min and 2 mL/min to maintain ≤20 g/L background concentration. Starting on the third day, samples were taken daily to monitor glucose and glycerol concentration, biomass accumulation, TFA, and oil profile.

After 238 hours, RT16 in vessel one consumed 364 g/L glucose and produced 80.1 g/L biomass and 72.7% TFA. EPA (4.9%), DPAn-3 (3.6%), and DHA (4.6%) were the omega 3 PUFA produced (Table 11 below). After 238 hours, RT16 in vessel two consumed 230 g/L glycerol and produced 84.6 g/L biomass and 61.1% TFA. EPA (7.0%), DPAn-3 (4.4%), and DHA (5.4%) were the omega 3 PUFA produced (Table 11 below). After 191 hours, RT16 in vessel three consumed 382 g/L glucose and produced 80.4 g/L biomass and 73.0% TFA. EPA (5.4%), DPAn-3 (3.5%), and DHA (4.3%) were the omega 3 PUFA produced (Table 11 below). After 238 hours, RT16 in vessel four consumed 258 g/L glycerol and produced 88.1 g/L biomass and 61.9% TFA. EPA (8.0%), DPAn-3 (4.7%), and DHA (5.8%) were the omega 3 PUFA produced (Table 11 below).

All conditions were useful for producing oil using RT16; however, Leiber H in combination with glycerol produced the highest % EPA (8.0%) and the highest mg EPA per g biomass (49.4 mg/g), while still achieving our targets of 55 g/L oil (55 g/L) and >50% TFA (62%).

TABLE 11
Fatty acid profiles expressed as percent of total fatty acids.

| Fatty | Vessel 1 | Vessel 2 | Vessel 3 | Vessel 4 |
|---|---|---|---|---|
| Acid | (Value %) | (Value %) | (Value %) | (Value %) |
| C14:0 | 5.4 | 4.1 | 4.8 | 3.3 |
| C15:0 | 3.5 | 5.9 | 3.6 | 6.7 |
| C16:0 | 15.2 | 18.4 | 15.4 | 15.6 |
| C16:1 | 18.8 | 10.6 | 18.5 | 10.1 |
| C17:0 | 1.3 | 1.8 | 1.4 | 2.3 |
| C17:1 | 1.8 | 2.7 | 1.8 | 3.3 |
| C17:2 | 0.5 | 0.9 | 0.5 | 1.2 |
| C18:0 | 1.1 | 0.9 | 1.1 | 1.0 |
| C18:1n-9 | 6.5 | 5.2 | 6.7 | 5.7 |
| C18:1n-7 | 10.1 | 9.2 | 10.4 | 8.5 |
| C18:2n-6 | 7.0 | 7.2 | 6.8 | 7.1 |
| C18:3n-6 | 3.6 | 4.3 | 3.9 | 4.1 |
| C20:3n-6 | 0.9 | 1.2 | 0.9 | 1.1 |
| C20:4n-6 | 4.3 | 4.7 | 4.5 | 5.0 |
| C20:5n-3 | 4.9 | 7.0 | 5.4 | 8.0 |
| C22:4n-6 | 3.4 | 2.9 | 3.1 | 3.0 |
| C22:5n-3 | 3.6 | 4.4 | 3.5 | 4.7 |
| C22:6n-3 | 4.6 | 5.4 | 4.3 | 5.8 |

### Example 11. Impact of Trace Minerals on Fermentation

To refine the levels of trace-minerals, specifically zinc, in the production media for RT16 fermentation performance for production of EPA containing oil, different amounts of trace minerals were compared. Specifically, 15%, 10%, and 5% of the control amount of zinc in addition to other modifications to the trace mineral amounts including twice as much manganese, and 20% the amount of cobalt, copper, and nickel as the control were compared. Vessel one contained control media, vessel two tested 15% Zn; vessel three, 10% Zn; and vessel four, 5% Zn. All three test conditions also had 200% Mn, and 20% Co, Cu, and Ni compared to the control media. All three test conditions produced more EPA % compared to the control, with vessel three (10% Zn) producing the highest (9.9% EPA).

Cultures of microorganisms of strain RT16 were pre-cultured in baffled 2 L Erlenmeyer flasks containing 500 mL of liquid media (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.36 g/L KCl, 0.1 g/L NaHCO₃, 0.8 g/L CaCl₂): 2H₂O, 0.12 g/L KH₂PO₄, 0.73 mg/L Na₂MoO₄·2H₂O, 2.3 mg/L NiSO₄·6H₂O, 24 mg/L FeCl₃·6H₂O, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 2.9 μg/L cobalamin (B12)). Flasks were incubated at 20° C. with 200 rpm of agitation for 4 days.

After the incubation period, 350 mL of the pre-culture was transferred into 3.15 L (10% v/v) of a media containing: 50 g/L glycerol, 9.66 g/L yeast extract (Leiber H), 6.44 g/L (NH₄)₂SO₄, 12.87 g/L monosodium glutamate, 3.4 g/L MgSO₄·7H₂O, 4 g/L NaCl, 2.7 g/L MgCl₂·6H₂O, 0.8 g/L CaCl₂): 2H₂O, 0.12 g/L KH₂PO₄, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, and 2.9 μg/L cobalamin (B12), in a 5 L fermentation vessel.

In addition to the above-described media, vessel one contained: 7.2 mg/L MnCl₂·4H₂O, 7.2 mg/L ZnSO₄·7H₂O, 96 μg/L CoCl₂·6H₂O, 4.8 mg/L CuSO₄·5H₂O, 4.8 mg/L NiSO₄·6H₂O, 24 mg/L FeSO₄·7H₂O, 2.4 mg/L Citric acid (anhydrous); vessel two contained: 14.4 mg/L MnCl₂·4H₂O, 1.08 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous); vessel three contained: 14.4 mg/L MnCl₂·4H₂O, 0.72 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous); and vessel four contained: 14.4 mg/L MnCl₂·4H₂O, 0.36 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous).

Batch culture conditions were applied as follows: 20° C., agitation at 400 rpm for the first 24 hrs. then increased to 600 rpm, aeration at 0.5 VVM (fixed and based on the starting volume) with atmospheric air, and pH 6.4-6.6. Fed-batch cultures were carried out for a total of 237.5 hours. Cultures were fed with a 100% glycerol solution, using a slow continuous pump rate between 0.06 mL/min and 2 mL/min to maintain ≤20 g/L background glycerol concentration. Starting on the third day, samples were taken daily to monitor glycerol concentration, biomass accumulation, TFA, and oil profile.

After 237.5 hours, RT16 in vessel one consumed 253 g/L glycerol and produced 76.6 g/L biomass and 61.9% TFA. EPA (7.4%), DPAn-3 (3.8%), and DHA (5.2%) were the omega 3 PUFA produced (Table 12 below). RT16 in vessel two consumed 264 g/L glycerol and produced 81.5 g/L biomass and 65.6% TFA. EPA (9.1%), DPAn-3 (3.7%), and DHA (5.0%) were the omega 3 PUFA produced (Table 12 below). RT16 in vessel three consumed 263 g/L glycerol and produced 82.8 g/L biomass and 64.9% TFA. EPA (9.9%), DPAn-3 (3.6%), and DHA (5.0%) were the omega 3 PUFA produced (Table 12 below). RT16 in vessel four consumed 268 g/L glycerol and produced 88.3 g/L biomass and 65.6% TFA. EPA (8.6%), DPAn-3 (3.4%), and DHA (4.4%) were the omega 3 PUFA produced (Table 12 below).

TABLE 12
Fatty acid profiles expressed as percent of total fatty acids.

| Fatty | Vessel 1 | Vessel 2 | Vessel 3 | Vessel 4 |
|---|---|---|---|---|
| Acid | (Value %) | (Value %) | (Value %) | (Value %) |
| C14:0 | 2.9 | 2.9 | 3.0 | 4.0 |
| C15:0 | 12.6 | 9.5 | 9.2 | 7.4 |
| C16:0 | 14.9 | 14.3 | 13.7 | 14.9 |
| C16:1 | 9.0 | 10.3 | 10.3 | 14.6 |
| C17:0 | 4.1 | 3.2 | 3.1 | 2.2 |
| C17:1 | 4.6 | 3.8 | 3.7 | 2.8 |
| C17:2 | 2.4 | 1.8 | 1.7 | 1.2 |
| C18:0 | 0.9 | 1.0 | 1.0 | 0.9 |
| C18:1n-9 | 2.8 | 3.3 | 3.4 | 3.6 |
| C18:1n-7 | 8.8 | 8.0 | 8.2 | 8.1 |
| C18:2n-6 | 5.1 | 6.0 | 6.0 | 6.4 |
| C18:3n-6 | 2.8 | 4.5 | 4.6 | 5.4 |
| C20:3n-6 | 1.1 | 1.1 | 1.0 | 0.9 |
| C20:4n-6 | 4.8 | 6.2 | 6.4 | 5.6 |
| C20:5n-3 | 7.4 | 9.1 | 9.9 | 8.6 |
| C22:4n-6 | 2.3 | 2.2 | 2.1 | 1.8 |
| C22:5n-3 | 3.8 | 3.7 | 3.6 | 3.4 |
| C22:6n-3 | 5.2 | 5.0 | 5.0 | 4.4 |

### Example 12. Examining the Levels of Vitamins and Trace Minerals in Seed Media

This example examines the levels of vitamins and trace-minerals in seed media for RT16 fermentation performance for production of an EPA containing oil. Comparing high and low levels of vitamins in two different seed media. Vessel one is the control seed media, vessel two had the same seed media as vessel one but with 4.8 times less vitamins, trace minerals, and potassium phosphate. At fermentation end-point vessel two produced ~0.5% more EPA than vessel one, with a slight (3 g/L) decrease in oil. Vessels 3 and 4 used a scaled down version of the fermentation production medium for their seed media, with 4.8 times less vitamins and trace minerals in vessel four than vessel three. At fermentation end-point vessel four produced the highest EPA %, 0.9% higher than control, but with a larger (10 g/L) decrease in oil. Vessel four and vessel two had comparable EPA % when comparing time-points with comparable oil titers. Essentially, changing the seed media conditions did not have much impact on the resulting oil composition at the end of fermentation.

Cultures of microorganisms of strain RT16 were pre-cultured in four baffled 2 L Erlenmeyer flasks containing 500 mL of liquid media as follows: flask one (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.8 g/L CaCl₂)·2H₂O, 50 ml/L 1 M PIPES buffer (pH 6.50), 0.36 g/L KCl, 0.1 g/L NaHCO₃, 0.12 g/L KH₂PO₄, 0.73 mg/L Na₂MoO₄·2H₂O, 2.3 mg/L NiSO₄·6H₂O, 24 mg/L FeCl₃·6H₂O, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 2.9 μg/L cobalamin (B12)); flask two (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.8 g/L CaCl₂·2H₂O, 50 ml/L 1 M PIPES buffer (pH 6.50), 0.36 g/L KCl, 0.1 g/L NaHCO₃, 25 mg/L KH₂PO₄, 151 μg/L Na₂MoO₄·2H₂O, 484 μg/L NiSO₄·6H₂O, 5 mg/L FeCl₃·6H₂O, 120 μg/L thiamine, 15 μg/L Ca-pantothenate, 15 μg/L nicotinic acid, 6 μg/L pyridoxine, 39 μg/L orotic acid, 7.5 μg/L biotin, 75 μg/L riboflavin, 150 μg/L pyridoxamine, 15 μg/L 4-aminobenzoic acid, 37.5 μg/L folic acid, 0.6 μg/L cobalamin (B12)); flask three (55 g/L dextrose monohydrate, 2 g/L yeast extract (Leiber H), 3.4 g/L MgSO₄·7H₂O, 4 g/L NaCl, 2.7 g/L MgCl₂·6H₂O, 2.7 g/L monosodium glutamate, 0.8 g/L CaCl₂)·2H₂O, 50 ml/L 1 M PIPES buffer (pH 6.50), 1.33 g/L (NH₄)₂SO₄, 120 mg/L KH₂PO₄, 14.4 mg/L MnCl₂·4H₂O, 0.72 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous), 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 2.9 μg/L cobalamin (B12)); and flask four (55 g/L dextrose monohydrate, 2 g/L yeast extract (Leiber H), 3.4 g/L MgSO₄·7H₂O, 4 g/L NaCl, 2.7 g/L MgCl₂·6H₂O, 2.7 g/L monosodium glutamate, 0.8 g/L CaCl₂): 2H₂O, 50 ml/L 1 M PIPES buffer (pH 6.50), 1.33 g/L (NH₄)₂SO₄, 25 mg/L KH₂PO₄, 3 mg/L MnCl₂·4H₂O, 0.15 mg/L ZnSO₄·7H₂O, 4 μg/L CoCl₂·6H₂O, 0.2 mg/L CuSO₄·5H₂O, 0.2 mg/L NiSO₄·6H₂O, 5 mg/L FeSO₄·7H₂O, 0.5 mg/L Citric acid (anhydrous), 120 μg/L thiamine, 15 μg/L Ca-pantothenate, 15 μg/L nicotinic acid, 6 μg/L pyridoxine, 39 μg/L orotic acid, 7.5 μg/L biotin, 75 μg/L riboflavin, 150 μg/L pyridoxamine, 15 μg/L 4-aminobenzoic acid, 37.5 μg/L folic acid, 0.6 μg/L cobalamin (B12)). Flasks were incubated at 20° C. with 200 rpm of agitation for 4 days.

After the incubation period, 350 mL of each pre-culture 1-4 was transferred into 3.15 L (10% v/v) of a media containing: 50 g/L glycerol, 9.66 g/L yeast extract (Leiber H), 6.44 g/L (NH₄)₂SO₄, 12.87 g/L monosodium glutamate, 3.4 g/L MgSO₄·7H₂O, 4 g/L NaCl, 2.7 g/L MgCl₂·6H₂O, 0.8 g/L CaCl₂): 2H₂O, 0.12 g/L KH₂PO₄, 14.4 mg/L MnCl₂·4H₂O, 0.72 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous), 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, and 2.9 μg/L cobalamin (B12), in a 5 L fermentation vessel numbered 1-4 respectively.

Batch culture conditions were applied as follows: 20° C., agitation at 400 rpm for the first 24 hrs. then increased to 600 rpm, aeration at 0.5 VVM (fixed and based on the starting volume) with atmospheric air, and pH 6.4-6.6. Fed-batch cultures were carried out for a total of 237 hours. Cultures were fed with 100% glycerol, using a slow continuous pump rate between 0.06 mL/min and 2 mL/min to maintain ≤20 g/L background glycerol concentration. Starting on the third day, samples were taken daily to monitor glycerol concentration, biomass accumulation, TFA, and oil profile. In vessel four, the agitation was increased to 750 rpm at 24 hours and then 800 rpm at 48 hours to maintain DO above 30% which was a struggle unique to this vessel only.

After 237 hours, RT16 in vessel one consumed 275 g/L glycerol and produced 91.4 g/L biomass and 67% TFA. EPA (8.6%), DPAn-3 (3.7%), and DHA (5.0%) were the omega 3 PUFA produced (Table 13 below). RT16 in vessel two consumed 272 g/L glycerol and produced 88.1 g/L biomass and 65.5% TFA. EPA (9.0%), DPAn-3 (3.7%), and DHA (5.0%) were the omega 3 PUFA produced (Table 13 below). RT16 in vessel three consumed 251 g/L glycerol and produced 85.7 g/L biomass and 63.7% TFA. EPA (8.1%), DPAn-3 (3.7%), and DHA (5.3%) were the omega 3 PUFA produced (Table 13 below). RT16 in vessel four consumed 248 g/L glycerol and produced 81.6 g/L biomass and 62.7% TFA. EPA (9.5%), DPAn-3 (3.6%), and DHA (5.3%) were the omega 3 PUFA produced (Table 13 below).

TABLE 13
Fatty acid profiles expressed as percent of total fatty acids.

| Fatty | Vessel 1 | Vessel 2 | Vessel 3 | Vessel 4 |
|---|---|---|---|---|
| Acid | (Value %) | (Value %) | (Value %) | (Value %) |
| C14:0 | 3.1 | 3.3 | 3.8 | 2.9 |
| C15:0 | 6.6 | 7.5 | 6.2 | 10.2 |
| C16:0 | 14.3 | 13.8 | 15.9 | 13.6 |
| C16:1 | 12.6 | 11.3 | 11.3 | 9.2 |
| C17:0 | 2.2 | 2.5 | 1.8 | 3.5 |
| C17:1 | 3.0 | 3.3 | 2.6 | 4.0 |
| C17:2 | 1.3 | 1.4 | 1.1 | 1.9 |
| C18:0 | 0.9 | 1.0 | 0.8 | 1.0 |
| C18:1n-9 | 3.8 | 3.8 | 3.7 | 3.1 |
| C18:1n-7 | 8.7 | 8.4 | 9.6 | 8.5 |
| C18:2n-6 | 6.5 | 6.5 | 6.3 | 5.6 |
| C18:3n-6 | 5.6 | 5.4 | 6.0 | 4.2 |
| C20:3n-6 | 1.0 | 1.1 | 1.1 | 1.1 |
| C20:4n-6 | 6.5 | 6.5 | 6.5 | 6.2 |
| C20:5n-3 | 8.6 | 9.0 | 8.1 | 9.5 |
| C22:4n-6 | 2.5 | 2.3 | 3.0 | 2.1 |
| C22:5n-3 | 3.7 | 3.7 | 3.7 | 3.6 |
| C22:6n-3 | 5.0 | 5.0 | 5.3 | 5.3 |

### Example 13. Testing Carbon Nitrogen Ratio in Fermentation Media

A carbon to nitrogen ratio of 27 in production media for RT16 fermentation for production of an EPA containing oil was examined. Comparing increased amounts of either monosodium glutamate (MSG) alone (vessel three), ammonium sulphate alone (vessel four), or MSG, ammonium sulphate, and yeast extract all together (vessel one) to increase the nitrogen content compared to the control media (vessel two) reducing the carbon to nitrogen ratio from 32 in the control to 27 in the three experimental media (vessels one, three and four). All three test conditions produced more EPA % compared to the control, with vessel one (increased MSG, ammonium sulfate, and yeast extract) producing the highest (10.8% EPA).

Cultures of microorganisms of strain RT16 were pre-cultured in baffled, 2 L Erlenmeyer flasks containing 500 mL of liquid media (55 g/L dextrose monohydrate, 6 g/L yeast extract (Leiber E), 3.4 g/L MgSO₄·7H₂O, 13.75 g/L NaCl, 2.7 g/L MgCl₂·6H₂O, 0.6 g/L monosodium glutamate, 0.36 g/L KCl, 0.1 g/L NaHCO₃, 0.8 g/L CaCl₂): 2H₂O, 0.12 g/L KH₂PO₄, 0.73 mg/L Na₂MoO₄·2H₂O, 2.3 mg/L NiSO₄·6H₂O, 24 mg/L FeCl₃·6H₂O, 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, 2.9 μg/L cobalamin (B12)). Flasks were incubated at 20° C. with 200 rpm of agitation for 4 days.

After the incubation period, 350 mL of the pre-culture was transferred into 3.15 L (10% v/v) of a media containing: 50 g/L glycerol, 3.4 g/L MgSO₄·7H₂O, 4 g/L NaCl, 2.7 g/L MgCl₂·6H₂O, 0.8 g/L CaCl₂): 2H₂O, 0.12 g/L KH₂PO₄, 14.4 mg/L MnCl₂·4H₂O, 0.72 mg/L ZnSO₄·7H₂O, 19.2 μg/L CoCl₂·6H₂O, 0.96 mg/L CuSO₄·5H₂O, 0.96 mg/L NiSO₄·6H₂O, 24 mg/L FeSO₄·7H₂O, 2.4 mg/L Citric acid (anhydrous), 576 μg/L thiamine, 72 μg/L Ca-pantothenate, 72 μg/L nicotinic acid, 29 μg/L pyridoxine, 187 μg/L orotic acid, 36 μg/L biotin, 360 μg/L riboflavin, 720 μg/L pyridoxamine, 72 μg/L 4-aminobenzoic acid, 180 μg/L folic acid, and 2.9 μg/L cobalamin (B12) in a 5 L fermentation vessel.

In addition to the above-described media, vessel one contained: 11.6 g/L yeast extract (Leiber H), 7.73 g/L (NH₄)₂SO₄, 15.44 g/L monosodium glutamate; vessel two contained: 9.66 g/L yeast extract (Leiber H), 6.44 g/L (NH₄)₂SO₄, 12.87 g/L monosodium glutamate; vessel three contained: 9.66 g/L yeast extract (Leiber H), 6.44 g/L (NH₄)₂SO₄, 21.34 g/L monosodium glutamate; and vessel four contained: 9.66 g/L yeast extract (Leiber H), 9.76 g/L (NH₄)₂SO₄, 12.87 g/L monosodium glutamate.

Batch culture conditions were applied as follows: 20° C., agitation at 400 rpm for the first 24 hrs. then increased to 600 rpm, aeration at 0.5 VVM (fixed and based on the starting volume) with atmospheric air, and pH 6.4-6.6. Fed-batch cultures were carried out for a total of 238 hours. Cultures were fed with a 100% glycerol solution, using a slow continuous pump rate between 0.06 mL/min and 2 mL/min to maintain ≤20 g/L background glycerol concentration. Starting on the third day, samples were taken daily to monitor glycerol concentration, biomass accumulation, TFA, and oil profile.

After 238 hours, RT16 in vessel one consumed 215 g/L glycerol and produced 89 g/L biomass and 64.2% TFA. EPA (10.8%), DPAn-3 (3.4%), and DHA (5.0%) were the omega 3 PUFA produced (Table 14 below). RT16 in vessel two consumed 190 g/L glycerol and produced 91 g/L biomass and 67.6% TFA. EPA (9.0%), DPAn-3 (3.7%), and DHA (4.9%) were the omega 3 PUFA produced (Table 14 below). RT16 in vessel three consumed 176 g/L glycerol and produced 75 g/L biomass and 58.7% TFA. EPA (10.0%), DPAn-3 (3.6%), and DHA (6.6%) were the omega 3 PUFA produced (Table 14 below). RT16 in vessel four consumed 271 g/L glycerol and produced 82 g/L biomass and 60.3% TFA. EPA (9.8%), DPAn-3 (3.4%), and DHA (5.7%) were the omega 3 PUFA produced (Table 14 below).

TABLE 14
Fatty acid profiles expressed as percent of total fatty acids.

| Fatty | Vessel 1 | Vessel 2 | Vessel 3 | Vessel 4 |
|---|---|---|---|---|
| Acid | (Value %) | (Value %) | (Value %) | (Value %) |
| C14:0 | 2.9 | 3.2 | 2.5 | 3.0 |
| C15:0 | 9.5 | 7.2 | 13.3 | 12.9 |
| C16:0 | 12.8 | 13.9 | 13.1 | 13.7 |
| C16:1 | 10.5 | 11.7 | 6.8 | 8.5 |
| C17:0 | 3.4 | 2.5 | 4.4 | 4.3 |
| C17:1 | 3.9 | 3.2 | 4.8 | 4.2 |
| C17:2 | 1.9 | 1.4 | 2.7 | 2.3 |
| C18:0 | 1.0 | 1.0 | 0.8 | 0.9 |
| C18:1n-9 | 3.1 | 3.9 | 2.1 | 2.2 |
| C18:1n-7 | 8.3 | 8.7 | 8.3 | 9.0 |
| C18:2n-6 | 5.8 | 6.7 | 4.3 | 4.2 |
| C18:3n-6 | 4.2 | 5.2 | 3.5 | 3.2 |
| C20:3n-6 | 1.0 | 1.1 | 0.8 | 0.8 |
| C20:4n-6 | 6.4 | 6.5 | 5.7 | 5.3 |
| C20:5n-3 | 10.8 | 9.0 | 10.0 | 9.8 |
| C22:4n-6 | 1.8 | 2.4 | 1.9 | 1.8 |
| C22:5n-3 | 3.4 | 3.7 | 3.6 | 3.4 |
| C22:6n-3 | 5.0 | 4.9 | 6.6 | 5.7 |

INFORMAL SEQUENCE LISTING

SEQ ID NO: 1:
Nucleic Acid Sequence of RT16 18S rRNA Gene
CTGGTTGATTCTGCCAGTAGTCATACGCTTGTCTCAAAGATTAAGCCAT
GCATGTCTCAGTATAAACAATTATACAGTGAAACTGCGAACGGCTCATT
ATATCAGTTATAGTTTCTTTGATAGTGTTTTTCTACATGGATACTTGTG
GCAAATCTAGAAACAATACATGCATCAAGGCCCGACTTTACGGAAGGGC
CGCATTTATTTGACTTAAACCAATACCCTTTGGGTTGTTTTGGTGATTC
AGAATAACTAAGCGAATCGCAGTGCCTTCGGGCGGCGATGAATCATTCA
AGTTTCTGCCCCATCAGTTGTCGATGGTAGGGTATTGGCCTACCATGAC
TGTTACGGGTGACGGAGAATTAGGGTTCGATTCCGGAGAGGGAGCCTTA
GAGACGGCTACCACATCCAAGGAAGGCAGCAGGCGCGTAAATTACTCAA
TGTTAACTCAACGAAGTAGTGACGAGAAATAACAATGCCGAGCCCTCAG
GGTTTGGCAATTGGAATGAGAGCAATGTAAAAACCTCATCGAGGATCAA
TTGGAGGGCAAGTCTGGTGCCAGCAGCCGCGGTAATTCCAGCTCCAATA
GCGTATATTAAAGTTGTTGCAGTTAAAAAGCTCGTAGTTGAACTTCTGG
TAGGAGTGACCCGGCCTTTGGGCGAATGCCCTCTGTTTGCTGTGTGTCA
TCTCTGGCCATCCTCGCTGTCTTTTAGGCAGCGTCTTTCACTGTAAAAA
AATTAGGGTGTTTCAAGCAAGATTATTTCTGGAATATATTAGTATGGGA
TGATAAGATAGGCTCTCGGTGCTATTTTGTTGGTTTGCACATCAAGAGA
ATGATTAACAGGGACAGTTGGGGGTATTCGTATTTACATGTCAGAGGTG
AAATTCTTGGATTTTGGAAAGACGAACTACTGCGAAAGCATTTACCAAA
GATGTTTTCATTAATCAAGAACGAAAGTTAGGGGATCGAAGATGATTAG
ATACCATCGTAGTCTTAACCGTAAACTATGCCGACTTGCGATTGTCCAA
TGGTTCTTTTAGCCGTGGGCAGCAGCACATGAGAAATCAAAGTCTTTGG
GTTCCGGGGGGAGTATGGTCGCAAGGCTGAAACTTAAAGGAATTGACGG
AAGGGCACCACCAGGAGTGGAGCCTGCGGCTTAATTTGACTCAACACGG
GAAAACTTACCAGGTCCAGACATAGGAAGGATTGACAGATTGAGAGCTC
TTTCTTGATTCTATGGGTGGTGGTGCATGGCCGTTCTTAGTTGGTGGAG
CGATTTGTCTGGTTAATTCCGTTAACGAACGAGACCACAGCCTACTAAA
TAGTATGTGCTATGGTAACATAGTATAAGACTTCTTAGAGGGACATTTC
GGGTTTACCGGAAGGAAGTTTGTGGCAATAACAGGTCTGTGATGCCCTT
AGATGTTCTGGGCCGCACGCGCGCTACACTGATGAATTCAACGAGTTTT
GCTCTTTGAGCATTTCCTTGGCCGGAAGGTCTGGGTAATCTTTTAAACG
TTCATCGTGATGGGGCTAGATTTTTGCAATTATTAATCTCCAACGAGGA
ATTCCTAGTAAACGCAAGTCATCAACTTGCATTGATTACGTCCCTGCCC
TTTGTACACACCGCCCGTCGCACCTACCGATTGAACGTTCCGGTGAGAC
CTTCGGAGCCTGTGTTTTTTCATTTATTTGAGAGTTCACGGGTAAAGTT
GAGCAAACCTTAACGTTTAGAGGAAGGTGAAGTCGTAACAAGGTTTCCG
TAGGTGAACCTGCAGAAGGATCA

What is claimed is:

1. A culture comprising
(a) a lipid-producing eukaryotic microorganism with an 18S sequence, wherein the 18S sequence has at least 100% identity to the sequence set forth in SEQ ID NO:1, wherein the microorganism has a fatty acid lipid profile; and
(b) a heterotrophic medium.

2. The culture of claim 1, wherein the lipid profile comprises 0%, or 0.1% to 19% of eicosapentaenoic acid (C20:5 n-3) (EPA) by weight of total fatty acids.

3. The culture of claim 1, wherein the lipid profile comprises 0%, 0.1% or 1% to 6% docosapentaenoic acid (C22:5 n-3) (DPAn3) by weight of total fatty acids.

4. The culture of claim 1, wherein the lipid profile comprises 0%, 0.1% or 1% to 45% docosahexaenoic acid (C22:6 n-3) (DHA) by weight of total fatty acids.

5. The culture of claim 1, wherein the lipid profile comprises 4% to 19% of EPA, 1% to 6% DPAn3, and 1% to 45% DHA by weight of total fatty acids.

6. The culture of claim 1, wherein the lipid profile comprises 10 to 30 milligrams of squalene per gram biomass.

7. The culture of claim 1, wherein the lipid profile comprises a ratio of EPA to DHA) between 1:1 to 4:1.

8. The culture of claim 1, wherein the heterotrophic medium contains vitamin B12.

9. The culture of claim 1, wherein the heterotrophic medium lacks vitamin B12.

10. The culture of claim 9, wherein the lipid profile of microorganisms of the culture lacking vitamin B12 in the heterotrophic medium has a higher pentadecanoic acid (C15:0) content by weight of total fatty acids compared to a lipid profile of microorganisms of a control culture containing vitamin B12 in the heterotrophic medium.

11. A method of making a lipid composition, the method comprising:
(a) culturing lipid-producing eukaryotic microorganisms with an 18S sequence having at least 100% identity to the sequence set forth in SEQ ID NO: 1 in a heterotrophic medium; and
(b) isolating the lipid composition.

12. The method of claim 11, wherein the heterotrophic medium contains vitamin B12.

13. The method of claim 11, wherein the heterotrophic medium lacks vitamin B12.

14. The method of claim 13, wherein the microorganisms of the culture lacking vitamin B12 in the heterotrophic medium have a higher pentadecanoic acid (C15:0) content by weight of total fatty acids compared to microorganisms of a control culture containing vitamin B12 in the heterotrophic medium.

15. The method of claim 11, wherein the heterotrophic medium contains between 1.0 to 30 g/L of a nitrogen source.

16. The method of claim 15, wherein the nitrogen source comprises at least two of yeast extract, monosodium glutamate (MSG), (NH₄)₂SO₄, urea, and NaNO₃.

17. The method of claim 15, wherein the nitrogen source comprises yeast extract, (NH₄)₂SO₄, urea, and NaNO₃.

18. The method of claim 22, wherein the nitrogen source is yeast extract.

19. The method of claim 11, wherein the lipid composition comprises 0%, 0.1% or 1% to 6% DPAn3 by weight of total fatty acids.

20. The method of claim 11, wherein the lipid composition comprises 0% or 0.1% to 19% EPA by weight of total fatty acids.

21. The method of claim 11, wherein the lipid composition comprises 0%, 0.1% or 1% to 45% DHA by weight of total fatty acids.

22. The method of claim 11, wherein the lipid composition comprises 4% to 19% of EPA, 1% to 6% DP An3, and 1% to 45% DHA by weight of total fatty acids.

23. The method of claim 11, wherein the lipid composition comprises 10-30 milligrams of squalene per gram of biomass.

24. The method of claim 11, wherein the lipid composition comprises a ratio of EPA to DHA between 1:1 to 4:1.

25. The method of claim 11, wherein the cultured eukaryotic microorganisms have a biomass accumulation of 2.0-150.0 g/L.

26. The method of claim 11, wherein the heterotrophic medium contains glycerol.

27. A method of using the lipid composition of claim 11 comprising incorporating the lipid composition into a foodstuff.

28. The method of claim 27, wherein the foodstuff is a human food, a pet food, a livestock feed, or an aquaculture feed.

29. An isolated biomass comprising lipid-producing eukaryotic microorganisms with an 18S sequence, wherein the 18S sequence has at least 100% identity to the sequence set forth in SEQ ID NO: 1, wherein the microorganisms in the biomass comprise fatty acids.

30. A method of using the biomass of claim 29 comprising incorporating the lipid composition into a foodstuff.

31. The method of claim 30, wherein the foodstuff is a human food, a pet food, a livestock feed, or an aquaculture feed.
